(19) United States
(12) Patent Application Publication
(10) Pub. No.: US 2025/0290079 A1
(43) Pub. Date: Sep. 18, 2025

(54) SYSTEMS AND METHODS FOR DEVELOPING AND USING BIOADHESIVES

(71) Applicant: Marvel Labs Inc., Marion, IN (US)

(72) Inventors: Nelson CHEPKWONY, Dedham, MA (US); Jake MILLER, La Fontaine, IN (US)

(21) Appl. No.: 19/079,047

(22) Filed: Mar. 13, 2025

Related U.S. Application Data
(60) Provisional application No. 63/564,727, filed on Mar. 13, 2024.

(51) Int. Cl. C12N 15/70; B33Y 70/00; C12P 19/04

(52) U.S. Cl. CPC C12N 15/70; B33Y 70/00; C12P 19/04

(57) ABSTRACT

Provided herein are systems and methods for producing and using bioadhesives. The bioadhesives may be produced in an Escherichia coli (E. coli) system. The bioadhesives may be used in a variety of different applications, such as industrial or medical applications. The bioadhesives may be used as a binder in Three-Dimensional (3D) printing.

### CROSS REFERENCE

This application claims the benefit of U.S. Provisional Application No. 63/564,727, filed Mar. 13, 2024, which is incorporated by reference herein in its entirety.

### BACKGROUND

Bacterial species belonging to Caulobacterales such as Hirschia baltica and Caulobacter crescentus use an adhesive structure, called holdfast, to adhere permanently to surfaces and form biofilms. Holdfast from these bacteria can act as a bioadhesive, with force of adhesion of 70 N/mm². Holdfast is composed of polysaccharides that are synthesized by these bacteria and modified to bind to a surface. Due to the low amounts that is produced by the bacteria, it is impossible to purify holdfast for use using conventional methods. Accordingly, there is a need for producing holdfast in larger quantities.

### SEQUENCE LISTING

The application contains a Sequence Listing which has been submitted electronically in XML format and is hereby incorporated by reference in its entirety. Said XML copy, created on Mar. 12, 2025, is named 65206-701.201 Sequence Listing.XML, is 70,754 bytes in size, and is incorporated by reference as if written herein in its entirety.

### SUMMARY

In some aspects, the present disclosure provides an engineered cell comprising one or more exogenous nucleic acid sequences that regulates production of a bioadhesive component, wherein the one or more exogenous nucleic acid sequences comprises at least 90% sequence identity to any one of SEQ ID NOs: 1-10 and 42-51.

In some embodiments, the one or more exogenous nucleic acid sequences encodes one or more proteins that regulate production of the bioadhesive. In some embodiments, the bioadhesive comprises a polysaccharide. In some embodiments, the bioadhesive comprises a holdfast component. In some embodiments, the one or more exogenous nucleic acid sequences are derived from Caulobacterales bacterium. In some embodiments, the Caulobacterales bacterium comprises Hirschia baltica bacterium or Caulobacter crescentus bacterium. In some embodiments, the one or more exogenous nucleic acid sequences are contained in a plasmid of the engineered cell. In some embodiments, the engineered cell comprises ten exogenous nucleic acid sequences with at least 90% sequence identity to SEQ ID NOs: 1-10 and 42-51. In some embodiments, the engineered cell further comprises a spacer sequence with at least 90% sequence identity to one of SEQ ID NOs: 33-35, wherein the one or more spacers are between one or more groups of the one or more exogenous nucleic acid sequences. In some embodiments, the engineered cell further comprises one or more promoters, wherein the one or more promoters comprise a sequence that is at least 90% sequence identical to any of SEQ ID NOs: 11-30 and 38. In some embodiments, the engineered cell is engineered from an E. coli competent cell. In some embodiments, the engineered cell comprises an E. coli BL21 (DE3) competent cell. In some embodiments, the one or more exogenous nucleic acid sequences is attached to one or more ribosome binding sites (RBSs).

In some aspects, the present disclosure provides a method for producing an engineered cell, the method comprising: (a) constructing a chimeric nucleic acid comprising one or more nucleic acid sequences comprising at least 90% sequence identity to any one of SEQ ID NOs: 1-10 and 42-51; (b) inserting the nucleic acid in a plasmid of a base cell to construct an engineered plasmid; and (c) transforming the engineered plasmid to an E. coli competent cell to generate the engineered cell. In some embodiments, the one or more exogenous nucleic acid sequences comprises ten exogenous nucleic acid sequences with at least 90% sequence identity to SEQ ID NOs: 1-10 respectively. In some embodiments, the nucleic acid further comprises one or more spacer sequences with at least 90% sequence identity to any of SEQ ID NOs: 33-35 between one or more groups of the one or more exogenous nucleic acid sequences. In some embodiments, the chimeric nucleic acid comprises one or more promoters, wherein the one or more promoter comprise a sequence having at least 90% sequence identify to any of SEQ ID NOs: 11-30 and 38. In some embodiments, the E. coli competent cell comprises an E. coli BL21 (DE3) competent cell. In some embodiments, b) comprises ligating the nucleic acid in the plasmid. In some embodiments, the one or more nucleic acid sequences regulates production of a bioadhesive. In some embodiments, the base cell does not produce the bioadhesive component.

In some aspects, the present disclosure provides a method for producing a bioadhesive, the method comprising: (a) providing an engineered cell (e.g., any engineered cell disclosed herein); and (b) subjecting the engineered cell to a medium that induces expression of the one or more exogenous nucleic acid sequences to produce the bioadhesive component. In some embodiments, the medium comprises Isopropyl β-D-1-thiogalactopyranoside (IPTG). In some embodiments, the method further comprises purifying the bioadhesive from the medium. In some embodiments, purifying comprises centrifuging and isolating the bioadhesive from the medium.

In some aspects, the present disclosure provides for a use of the holdfasts and bioadhesives discussed herein. In some embodiments, the bioadhesive is used as an adhesive in one or more applications. In some embodiments, the application is Three-Dimensional (3D) printing. In some embodiments, the bioadhesive is used as a binder in 3D printing.

In some aspects, the present disclosure provides for a bacteria cell that has been modified to secrete a bioadhesive faster and/or in higher quantities than an unmodified bacteria cell. In some embodiments, the wildtype bacteria cell comprises an E. coli cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of holdfast binding to glass surfaces, the process of isolation of holdfast from cell culture, purification with different concentrations of salt, and quantification by microscopy.

FIG. 2 depicts an exemplary workflow for the method of producing holdfast polysaccharides using E. coli cultures. The steps involved include growth, induction, purification, filtration and harvesting of polysaccharides.

FIG. 3 depicts an exemplary engineered pET28 plasmid-nucleic acid construct.

FIG. 4 depicts an exemplary engineered pET28 plasmid that is used for production of holdfast in E. coli, comprising genes for HfsA, HfsB, HfsC, HfsF, HfsE, HfsG, HfsL, HfsJ, and HfsH among others.

FIG. 5 depicts an exemplary pET-Orl-Hfs plasmid that is used for production of holdfast in E. coli, comprising genes for HfsA, HfsB, HfsC, HfsD, HfsE, HfsF, HfsG, HfsL, HfsJ, and HfsH.

FIG. 6 depicts the process whereby non-sticky or non-adhesive polysaccharides are converted into sticky or adhesive polysaccharides through hfsH mediated deacetylation.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The term “about,” as used herein, with reference to a number refers to that number plus or minus 10% of that number. The term “about” a range refers to that range minus 10% of its lowest value and plus 10% of its greatest value.

The term “bioadhesive,” as used herein, refers to a biologically derived adhesive substance. The bioadhesive may be used by one or more organisms to adhere (e.g., permanently adhere) to surfaces and form biofilms. The term “bioadhesive” may also refer to an artificial bioadhesive produced by the systems and methods disclosed herein.

The term “holdfast,” as used herein, refers to a specific bioadhesive of an organism used for the purposes disclosed herein. It may also refer to an artificial holdfast produced by the systems and methods disclosed herein.

### Systems

Bioadhesives are materials that can adhere to different surfaces. Many bioadhesives are natural polymeric materials, which can act as adhesive in order to adhere to many different surfaces in various applications. For example, the bioadhesives disclosed herein may be used in medical applications, such as: tissue adhesives, hemostatas, tissue sealants, functional wound dressings, medical device fixation, medical sutures (e.g., a replacement for medical sutures), and a replacement for traditional drug dosage systems. For example, the bioadhesives disclosed herein may be used in industrial applications, such as tapes, glues, superglues, drywall, plywood, oriented strand board (OSB), plumbing, and three-dimensional (3D) printing. For example, the bioadhesives may be used as a binder in 3D printing.

In some embodiments, for 3D printing, holdfast (e.g., a bioadhesive produced by the methods and systems described herein) may be jetted or dispensed from a printhead or nozzle(s) onto the material intended to bind together. This can be achieved through a traditional binder jetting process or through a process involving ultrasound to form the material into the layer pattern, and a nozzle dispensing the holdfast to bind the layer, repeating the process until the desired end result is achieved. Holdfast can also be used as a general binder (glue) or adhesive sealant in medical applications, such as: tissue adhesives and bone repair, hemostats, tissue sealants, functional wound dressings, medical device fixation, medical sutures (e.g., a replacement for medical sutures), and a replacement for traditional drug dosage systems.

In some embodiments, the bioadhesives disclosed herein may be harvested using one or more biological systems. As an example, the present disclosure provides for Escherichia coli (E. coli) systems to synthesize the bioadhesives discussed herein. E. coli is a bacterium that can be used to produce proteins, store deoxyribonucleic acid (DNA) sequences, and test protein function. E. coli is useful for these purposes because it grows quickly, is easy to manipulate, and is cost-effective. Therefore, the E. coli systems disclosed herein may provide a cost-effective method for mass producing the bioadhesives disclosed herein for their different applications.

In some embodiments, the disclosure provides an engineered cell for expressing and synthesizing the bioadhesives disclosed herein. In some embodiments, the bioadhesive comprises a holdfast. In some embodiments, a holdfast is a bioadhesive used by an organism to adhere (e.g., permanently adhere) to surfaces and form biofilms. In some embodiments, the holdfasts used by these organisms can be artificially synthesized using the systems and methods provided herein for a variety of medical and industrial applications disclosed herein.

In some embodiments, the engineered cell may comprise a bacteria cell that has been modified to secrete a bioadhesive component faster than a wildtype or unmodified bacteria cell. In some embodiments, the wildtype or unmodified bacteria cell comprises an E. coli cell.

In some embodiments, the engineered cell may include one or more exogenous nucleic acid sequences that regulates production of a bioadhesive (e.g., holdfast). In some embodiments, the one or more exogenous nucleic acid sequences may have at least about 70%, at least about 75%, at least about 80%, at least about 85%, at least about 90%, or at least about 95% sequence identity to any one of SEQ ID Nos: 1-10 disclosed in Table 1. In some embodiments, the one or more exogenous nucleic acid sequences may have at least about 70%, at least about 75%, at least about 80%, at least about 85%, at least about 90%, or at least about 95% sequence identity to two or more sequences of SEQ ID Nos: 1-10 disclosed in Table 1. In some cases, the one or more exogenous nucleic acid sequences may be any one of the sequences disclosed in Table 1. In some embodiments, the nucleic acid sequence includes one of the genes disclosed in Table 1. In some embodiments, the nucleic acid sequence includes two of the genes disclosed in Table 1. In some embodiments, the nucleic acid sequence includes three of the genes disclosed in Table 1. In some embodiments, the nucleic acid sequence includes four of the genes disclosed in Table 1. In some embodiments, the nucleic acid sequence includes five of the genes disclosed in Table 1. In some embodiments, the nucleic acid sequence includes six of the genes disclosed in Table 1. In some embodiments, the nucleic acid sequence includes seven of the genes disclosed in Table 1. In some embodiments, the nucleic acid sequence includes eight of the genes disclosed in Table 1. In some embodiments, the nucleic acid sequence includes nine of the genes disclosed in Table 1. In some embodiments, the nucleic acid sequence includes ten of the genes disclosed in Table 1. In some embodiments, the nucleic acid sequence includes twelve of the genes disclosed in Table 1. In some embodiments, the nucleic acid sequence includes thirteen of the genes disclosed in Table 1. In some embodiments, the nucleic acid sequence includes fourteen of the genes disclosed in Table 1. In some embodiments, the nucleic acid sequence includes fifteen of the genes disclosed in Table 1. In some embodiments, the nucleic acid sequence includes sixteen of the genes disclosed in Table 1. In some embodiments, the nucleic acid sequence includes seventeen of the genes disclosed in Table 1. In some embodiments, the nucleic acid sequence includes eighteen of the genes disclosed in Table 1. In some embodiments, the nucleic acid sequence includes nineteen of the genes disclosed in Table 1. In some embodiments, the nucleic acid sequence includes twenty of the genes disclosed in Table 1. In some embodiments, the nucleic acid sequence includes all twenty-one of the genes disclosed in Table 1.

In some embodiments, the one or more exogenous nucleic acid sequences may have at least about 70%, at least about 75%, at least about 80%, at least about 85%, at least about 90%, or at least about 95% sequence identity to any one of SEQ ID Nos: 42-51 disclosed in Table 1. In some embodiments, the one or more exogenous nucleic acid sequences may have at least about 70%, at least about 75%, at least about 80%, at least about 85%, at least about 90%, or at least about 95% sequence identity to two or more sequences of SEQ ID Nos: 42-51 disclosed in Table 1.

TABLE 1
Nucleic Acid Sequences

| Gene | Sequence | SEQIDNO: |
|---|---|---|
| hfsE | ATGTTTAAATTTGTAATGGAAATCGCTGGCATGAGCCTCGACG | 1 |
| | CTATGGATCCGGACAATAATGCAAGTAACGATACATATCGTAT | |
| | TGCAAAATTGCGCCAGCACAAAAGCGAAAAACACAAATTACA | |
| | CAATACACAACCTGATCAACCCGCCAAACGTTTTCCAATAGAT | |
| | CGCTCTGGTCTACGTTTTATACTGCGCATATTTGATTTTACCGCC | |
| | GGCTTTGCAATTATTCTTATTGCTTGTCATCTTTTAAAAATTGAT | |
| | CTGCTAAGCGAAAACCTTAGAACTTCTCTGCCCTACATAGCAG | |
| | CGCCATTATTTGCCTTCTTGGGCATGCGTATAACGGGTGCCTAT | |
| | CGTTTTGCATTTGATGAACATCCACTTACACACATTGGACGTGT | |
| | TTTGCTTGGCTCAAGCCTTGGGGTGGCACTTGTTCACATCATTA | |
| | GTTTGGTTTTCAATCTTGGTGATGATCAGCTTTACCAAATTTCT | |
| | GCAATTGTTTTGTGTGGCTATTTGGGATTGCACGCGCATTACGC | |
| | CGCTCTTCTTCGTTCCCTCACGCGTTCTGGCGCGCTTGCAGATA | |
| | ATGTCGTTATCGTCGGTGCGACCCCTGCTGCTTTTGATTTAATC | |
| | GAAAAAAATCGCAAAAAACGTGAGATGAATATTCTAGGTGTCT | |
| | TTGAAGACAGGCTAGATCGCGCACCCGCAGCAATCGCGGATGT | |
| | TCCGGTCATTGGGAAAGTCGATGATCTACTCGAATGGGATAAA | |
| | CTTCCTGATGTCGACAGAATTATCCTAACAGTGACATCAACCGC | |
| | ACAAGAACGTGTTCGTACCCTTATAGATCGTCTACGCCTTTTGC | |
| | CTCAAGAAGTGATTCTCATGCTGGACCTAGACGGGTTTAGTCC | |
| | AGAGAAAACCTCAATCGCCAATATGGTCGATACACCCGCAGCT | |
| | TATGTTTCAGGTGCGCCCAAAGATGTCAGACGTGCCGCGATCA | |
| | AACGCGGTTTAGATATTGTGGTTGCAAGCGCCATGCTCATACTT | |
| | TTCTTGCCATTTATGATGATCATTGCAACTTTGATCAAACTAGA | |
| | TAGCCAAGGCTCAATTTTCTTTAGACAGCGACGTCATGGTTTTA | |
| | ACAATCAGATTATTCGTGTCTGGAAATTTCGGACTATGCACCCG | |
| | GATAAAGCGGCTGAAGATGGCACAAAAGTCATCCAAACCGTG | |
| | AGCAATGATAAACGTGTCACGCGCATTGGTGCCTTCTTACGTCG | |
| | CACAAGTTTGGATGAACTTCCCCAACTCATAAATATTCTAGTTG | |
| | GAGACATGTCACTTGTCGGCCCTCGCCCCCATGCCGTTGGCATG | |
| | ACAACCGAGGAAGTTGAAGTCCACAATATCGTCGCTGAATACG | |
| | CTCATCGTCACAGAATGAAACCAGGCTTAACCGGTTGGGCACA | |
| | AATCAATGGATCTCGCGGCCCAGTCCACACAGCAGAGCTTGTT | |
| | AAAGATCGCGTGCGTTTGGACATGGAATATATGGAAAAAGCAT | |
| | CATTCTGGTTTGATCTATACGTCATCCTGATGACTGCTCCCTGC | |
| | CTTTTGGGCGACTCAAAAACACAGCGCTAG | |
| hfsG | ATGAACACAACGCCCCAACTTAGCGCCAACCCACCCGTTCGCG | 2 |
| | ACACGACAACCCCACGCGAAATCGTGGTCGATAACCCGCACTG | |
| | GAAAGACAAGGCAAACCAAAAGCTGTCTATTCTTGTACCTAGC | |
| | TATAAAGACGATCCAGAAGCTTTATTGGCCTCATTATCCAAATG | |
| | CAAAAATGTCGAAGACATCGAATTTATTTTTATGATGATGGCG | |
| | GCGGAGACCGCACACTTATTGAGAAAATTTCGGCGCACGCAGC | |
| | TGAGGCCAGCTTTCCAGTTCGGATCATCTCTGCACAACACAATA | |
| | TTGGACGTGCAGGCGCGCGAAATCGTTTATTGAACTATGCGCG | |
| | ATGCAAATGGGTATTGCTCCTTGATGCCGACATGCTTCCCGATA | |
| | ACGAAGATTTCCTGACTAATTACATCCAAGAAATATCAGTTCAT | |
| | CCCGACCCTAAACTTATCGTCGGCGGGTTTTCACTTAAGCAAGC | |
| | CTCAACGGCCCCAGAATATGCTTTGCATCGGTGGCAAGCTGAA | |
| | AAATCAGAATGCATGTCTTCTAAGCTGCGCAACACAGAACCTG | |
| | GGCGTTATGTTTTCACAAGTAATGTTCTTGCCCATGCAGACCTG | |
| | TTTGAAGAAGTCCCTTTTGATGAAACATTCAGTGGATGGGGCT | |
| | GGGAAGATGTCGATTGGGGATTGCGTATCGCCAAGACTTATCT | |
| | AGTTCTCCACATAGATAATACAGCCACTCATTTAGGCCTTGATA | |
| | CTGATCGAGATCTCATGAAAAAATATGGCAAATCGGGTGCTAA | |
| | TTTTAAAAAAGCGATTGAAAAACATCCCGACGCTTTAAAATCA | |
| | ACAAGTCTATATAAAGTTGCTAACAAGCTAGCGCCGATACCAT | |
| | TCAAGCCCGTGATCAAATCCATAACTGGTAGCATTGCGACAGC | |
| | GCACTTTTTTCCAATAAAGGTTAGAGGTCTCGCCCTAAAATTAT | |
| | GGCGTGCAACCATATATGCGGAGGCTCTTCATGATTGA | |
| hfsL | ATGAACCAATCCGTAAAAGTCAGTATTGTTATTCCAACATTTCG | 3 |
| | CCGCAAGACGGTTTGGAAAATGCCATTACCAGCATTCTGAATA | |
| | TGACGGATGCTGCTTTGGAAACGGCTGAAATCGTTGTCGCAGA | |
| | CAACTCACCCGAAGCCGGCGCCAAATCCACAGTAGAACAACTC | |
| | AAACTCAACACTAGCGTACCAATAAACTATGTCAGTGAACCCA | |
| | CTCCGGGCGTATCAAATGCACGAAATGCTGGTCTAGCTGTAGC | |
| | AAATTCTCGTCTAATTGCCTTCATTGACGATGATGAAACTGCCC | |
| | AAAATGGATGGCTAGATAATTTAGTCGCAGCTCATCAAAAGCT | |
| | CGGTGCCGCTGTCATTTTTGGCCCTGTCGAAACTGTGCTTCCAA | |
| | CCGACACGCCTCCCGCGCACAAACAATATCTGGAAGATTTTTTC | |
| | TCTCGCAAAGGCCCAAGCGAAACCAAACTAATTGATGAAGCTT | |
| | TTGGCTGTGGAAATGCTTTCCTTGATTTGGATCGCATTCAACCT | |
| | TGCCTCCCTGAAAATGAGCCGTTTTTCAATAAGATAGCCAATG | |
| | AAACTGGCGGCGAAGATGACTATCTTTTTGCTCGTGTGAAACA | |
| | GCATGGTGAAACATTTGGCTGGGCACATAATGCAATTGTTGAT | |
| | GAACACGTCCCCGAAAAACGTGCACATTTAGGTTATACACTGA | |
| | GGCGTGCATTCGCCTATGGACAGGGGCCCTCCACCAATTGCTG | |
| | GCGCAATAATGACGGCATTGATATTCCACGCCTATTGATGTGG | |
| | ATGATTATCGGGACAGGTCAGTTTATTATCTATGGAATCTCTGC | |
| | CTTAGCGATGTTTGCAATCAAGCATCCCAAACGCGCATATATGT | |
| | TGGATAAAGCTGTAAGAGGACTAGGAAAACCCCTCTGGTTTCC | |
| | ACCATTTATTCTGCAATTTTATGGGGATATTCCCGATAAGAAAA | |
| | AGCGCAAAAAAGTTATCAAAAGCGCAACTTAA | |
| hfsJ | ATGTCGTCCGGTAAGAAAATTTACGACATGCTAGAACATTTAA | 4 |
| | CGGTTCCTGAAACGGAAGCGAATGTGGATGCATTGCTAAATGG | |
| | ATTGCCGGATGCGACTTCACCTCAGGTGATTTCGTTTGTGAATG | |
| | CACACGCTGTAAATTTGATGGTGAAAGATGAGGGCTTGTTCAA | |
| | AGCGCTGATCGGATCGGATATTTTATTGCGAGATGGTTCAGGC | |
| | ATGAAGATTTTAATGAAATGGCTAAACCAAAATCCGGGTGCAA | |
| | ACCTGAATGGTACAGATCTAATACCACGCATTATTGAGAAATT | |
| | TGATGGTATGAAGGTCGCTGTATTTGGAACTCAGGAGCCTTGG | |
| | TTGTCTAAAGGCTGTGATGTCATTGAAACGCGTGGTGGAACAA | |
| | TCGTATCGCGTTTGAATGGTTTCCAAGATGAAGCAGCTTATATT | |
| | GAAGCGATTGAGACCTCTAAACCCGACCTTGTGATTCTTGCTAT | |
| | GGGAATGCCCAAGCAAGAAATGACTTCTATGGCTTTGCGGGCA | |
| | GCAGCGAGCTGGCCGACGACAATAGTAAATGGTGGAGCTATTA | |
| | TTGATTTCCTTGCTGAACGCGTTAACCGCGCACCAGAAACTTGG | |
| | CGAAAGCTGGGTATGGAATGGCTCTACCGTCTCATTCAAGAAC | |
| | CTAAACGACTATTCGGCCGATACGTGGTTGGAAATGTCATTTTT | |
| | CTCACGCGTGGTTTGATATTATGTGTGACCCAAGCTAATCCCAA | |
| | AATAACTTAA | |
| hfsH | ATGATTGATTGGCATTACACACCCTCTCGTACCCTTCCCGCCAA | 5 |
| | ATTAAAACGCCGCATGACACAATGGAGACATGCTGCGCCAGTT | |
| | GATGTGAGCAACACACAATTTCATGTATCATACACATTTGATG | |
| | ATTTTCCAATGTCGGCGGTCAATGGTGCCGACATCCTCGAATCT | |
| | CATGATGGGCACGCAGCATTTTATGCCTGCACAAAAATGATAG | |
| | GCACACACGGCGCATATGGGGACATGTACGATATCAAAACCAT | |
| | GTTGGACCTTGAAAACCGTGGACATGAAATTGGCGCGCATACC | |
| | CACAGTCACCTTGATTGCGCGCAAAGCAAACGTGAAACTGTCT | |
| | TAAATGATATTGACGCAAATATTTCTGCGCTCATGGAAGCCGG | |
| | CTTAAAAAAACGGCCCACAAGTTTTGCATACCCTTATGGAGAA | |
| | ACCCTTTTTGATACCAAAAAAGAAGTATTCAAAAAGTTTGATCT | |
| | GTGTCGCGGTATTCTTCCCGGAATTAATGTTGGAAAAGTTGATC | |
| | TCGCTCAATTGCGGTGTTTTGAACTAAACGAAAATCCAGCCAC | |
| | ACGCATTCGAGCAATAAATGCTATCGAAGAAGCAGGCAAAACT | |
| | GGCGGCTGGGTGATTATATTTACACATGATGTATCACCTCAGCC | |
| | AACTGCGTATGGAACCACAACTGGGATCGTCGAAGAATTGTGC | |
| | CAACTGTCTAAAGCAGCAGGCGCAACGCTTTCAACGCCAACTG | |
| | AGGCAGCAAGAAGCTACGGCCTAATCTCATGA | |
| hfsF | ATGATGCAAGACAATCAAGCCACCCAAATGCCTGTCCGGCGCG | 6 |
| | AACAACGCGGTGTGAATTTACGCGTTCGTCCACGTTTTGGCGTG | |
| | GGCGATATATTCTTGCAATTGTGGCGATCACTTTGGATTATGAT | |
| | ATTGGTGTTTCTGCCTATCGTGATACTGGGTGTTCTGTTTGCTAT | |
| | GACCATGCCCAAAACATATACGGCATTCGGCACAATGCAGGTC | |
| | ACGTTGGATAAGCAATATATTTATGACCCGCTTGTCGGAGATG | |
| | CTGGACGTGGTGTTTCTATTGAAACTGAGGCAATTGTTAGTGCG | |
| | GAAGCTGAGAAAGCCAATTCCCCTATATTAGCGCGTCGCGTTA | |
| | TGGATCAGATGGGGATTGGACACATCTATCCTAAAATTGCCCA | |
| | AGAGATAGCAGAAACGAATGACCCTGACAAAATTCGAAAATT | |
| | GGAAGCCTCTGCATATGATGCTTTGAACCAAAATTTTGGTGCAT | |
| | CTCACGGGGTGAAATCACCCTTGCTTACTTTTGTCTTTAAGCAT | |
| | GAAGATCCAATTGTTGCCGCCGAGGTTGTCAATAAATTTCTGG | |
| | AACAATTTGTCGACTATAGAGAAATTCAAAGTGATCAAGATGA | |
| | TATTGCGGCTGTGGCTGGTCAAAGATATCTTGTGGGTGAGGCCT | |
| | TAAGTGAAGCCGAATCATTGCTACGCGACTTCCTTGTTGAAAAT | |
| | GAAATTGGTGAATTTGATACTGATCGATTGTCTGTTGGCACGAC | |
| | ATTGGTGAATTTACGCAATGAACTTTTGACCGTTGAGGCATTGG | |
| | TAAAAGAAGGTGAAGGTCGTTTGAGTGGCTTGCGTGCTATGCT | |
| | GCCAACTACGCCAGAAACGATTGCGCTAGAAGTTGAGACAAAT | |
| | GCATCACAACGTGTGCTGGACCTCCAGTTAGAGCGTGAGCAAT | |
| | TATTGCTAAGATATTTGCCTGATAGCCGCGCTGTTTTAGAAATA | |
| | GATGCGCGTATAGCGAGTATGAAATCACTGTTGGAGAGTGATG | |
| | ATGGCGGTGTTCGTCGAACTGGTCCAAACCCAGCGTATCAAGA | |
| | ATTAATCTCTACAATCACGAATGTTCAATCTGATTTGAATGCGG | |
| | CAACGTCTCGCGCGGCGGAGTTAAAACGTCAGGTCAATGAAGT | |
| | GTCTCAGCGACAGAAAGAACTCGTTTTGTTGCAACCAGAATAT | |
| | AAGAAACTTCGCCGAGAGCGTGACGTGTTGGAAAGAGCGATG | |
| | GTTGAATTGGCAACGAGAGAGCAATCGAAAAAAGCAGAAGTT | |
| | CAGATTTCGGAAGGTTCAAGCGGGAGTGTTGTAATCGTCGATA | |
| | GGGCATTACCACCATCTAAAGGTTCGAGTATGAAGATACCGAT | |
| | TGTTTTGGCATCAGGGATGTTTGCTGGATTTACTGCATTAATCG | |
| | CGGGCTTACTTGTTGCATTTAGTCGTAAAAGTCTCTCAACGCGG | |
| | CGTTCAACTGAAAAGACAATAGGGTTGCCTGTCATTGGTGTGA | |
| | CATCGAAGCAATAA | |
| hfsC | TTGTTCAACAACGACACGTGCCTTTCAAAATGGCACAACAGCC | 7 |
| | CACATTCATTAAATGGACCTCTGTACTGGATCGAACGTGCGTTA | |
| | GTTTTCTTCATTGTTTTAACATACTCAGGACTATGGATTGCGAT | |
| | ACTCTTAGGTAGAGCCGCAGAAACACAAAGCATGATAGCACCT | |
| | GACCCAGGACCAATAGCGCGGGCTTCTTGGTTTCCTGCATATCT | |
| | GGCATTGATTGGGTTATTGGGGCTAAATATAAAAAAGCTAGGA | |
| | AGTGCGAGCTTAAAATTCTGGCCCATAATTTTGTTATTAGGTTT | |
| | AGCAGTCGCTTCGGCAAACTGGTCTTTAGATCCAAGCCTAACA | |
| | CAGCGGCGTGCCATTGCACTTAGCTTCAGCTTTATGTTCGGTTG | |
| | TTATCTTGCGATACGTGCCCCCCTAGTCGACACACTAAGGATCA | |
| | TTGGCTGGGCGTGGCTAACGATTTGTATCCTCAATTTTCTTCTT | |
| | ATTTTTGGAGCCCCTCACCTAGGTGTCCACTCGGAATTACACGT | |
| | TGGAGCGTGGCGCGGTTTCATGACCGAGAAAAACCATCTTGGC | |
| | GGTGAAATGGCACGCGCAAACCTAATATTCCTTGCGCTTGTAT | |
| | ATTTCGATAGAAAAACACCAGCAGGTCAGAACAAAAAAGCAT | |
| | GGTGGTTAGGTCTAGCTTTAACGTGGATGCTCATCTTAGGTTCC | |
| | ACATCTAAAACGGCGCTCATTGCAACGCTAATCCCATATTTAG | |
| | GTTTTTTATTCTACAGCATCGCGATCCGCACACCGATATTAGGA | |
| | CTTTTAGCGTTTTGGGGTGGCCTGAGTTTAGCTGGTATCGGTTA | |
| | CGCCATAATATCCATTTCGCCTGAAACTGTTGTTGCGCTGATTG | |
| | GGAAAGACCTGACGTTTACCGGTCGGACAGGCATTTGGGCTAT | |
| | CGTTATTGATTTAATCAATCAGCAAAAATGGACGGGTTATGGA | |
| | TATGGTGCATTCTGGGTCACGCCTGATGGTCCAGTTGCATTTAT | |
| | CGTGAACACACTGGAATGGAACGTGCCAACAGCCCATAATGGA | |
| | TGGCTGGAAGTGGGATTGGCTATAGGATACCCTGGATTAATCC | |
| | TGATTATCTGCATCTCACTATTTGCTTTAGGCAAAGCTGCATAT | |
| | CTGGCAACAGGCAAACATGGCCCATTTGTCTTTCTCATGCTTTT | |
| | TCAAATAATTCTATTTTCCTTGTCAGAAAGCATATTGATGCAAC | |
| | AAAATTCGCATGCATCATCCCTATTCTATTTCTTCACTGCTTAC | |
| | GCATTTATTGCTCGCAAAGTCGCAACAGACTCACAAACACCTC | |
| | TCTTAAGCGCCCCGCATTGGGCGCTACCACCGCGTACTACGAA | |
| | GAAACGCAGCTAA | |
| hfsD | ATGATGAAAAACAACGCATCCATCCTCGCACTCGCTGTTCTGTC | 8 |
| | AATATCGGCATGCTCTAGCCCTCAACCGGGACCAAGCGCGCCG | |
| | ACTTTTCAATCAACGACTTTCTCAAAATGGTCGCAAAATGATGC | |
| | AGCCTACAGATTCTACCCAGGCGATAAGTTAAACATAACTTTC | |
| | AGACAAGCACCTGAATTGGACCGAGAAGTCGTTATTGCACCGG | |
| | ATGGTCGGATAAGCCTTCCACTCATGGACCCTGTCGTTGTCGCG | |
| | GATCTTTCTGCTTTCGAACTACAAAAAATTCTTGAACGCATCTA | |
| | TGCTAGAGAATTGGTGGATCCCTCTCTCACAGTCACGCCAGTTG | |
| | AATTTGCGTCCCAACAAATATTCGTCGGTGGTGAAGTCAATAA | |
| | TCCGGGCGTATTTCCATTGCCGGGCCAAATTGATCCGCTGCAAG | |
| | CCATTGTCTTAGCTGGTGGCTGGAACGATAATTCCAAACCTGA | |
| | ACAAGTCATTATTTTACGTCGCGATCGAAATGGTCAAATCATG | |
| | ACCCGCGTTGTTGACGTAAAAAATGCTCTTCGTGACCCAAGCA | |
| | ATCTAGACATTGGACCATTAAAACGGTTTGACGTCGTGTTTGTA | |
| | TCCCGCAGCCGCATAGCAAATGAAAACAAATTTATACAGCAAT | |
| | ACGTACTATCAGCATTGCCAATTGATTTTTCATTCTTCTACAAC | |
| | CTAAAAGACAACGCCTTCTAA | |
| hfsB | ATGAAAGACTTACGAAAAGATTTAACTGAAACTTGGCGTGTCG | 9 |
| | CAACACGAGCACCAGTGGATAATGGTGGTCGTACGATTATGTT | |
| | CATGTCTGCTATGGCAGGCGAAGGCACATCGAGTGTGGCTGCT | |
| | TCTTTTGCTATGTTAGCGGCTCAACGTGCGCGCAAGGGTGTATG | |
| | GCTGATTGATCTAAATCTTATGGATGGTCGGTTATTTAATGCTT | |
| | TTGATCGCGGCGGCGATTTTGTTGATTCATTTGGTCAGGTTGGT | |
| | CCAGCTCATAACGCTGAAATGTCTGGCGCTTCCTTTTTTTCTAT | |
| | CTCACCACCACCACCGCCGCCACCACCTGGTAAGAAGAACGAT | |
| | GCGGGGCTTTTTGTAATGCATCGTGTGGCGGATACTAAATTACT | |
| | TGTGTCTAGGTTTCGAAAAGAACGTTTAGAATCAAGCGCTCGC | |
| | GTGCGTGTGAAAACGGGAGCGGACTACTGGAAAGCCGTTAGA | |
| | GAGATAGCTGACTGGGTCATTATCGATGCACCTGCGTTAGAAA | |
| | CATCTTCAGCGGGTTTGGCTATCTGTTCGCAAATGGATGCAACT | |
| | GCTTTAGTTGTGCGAGCTGATAAAACACCTGCGACTCAGGTGT | |
| | CCAAGCTTGGACAAGAAATTGAAGGCCATGGTGGCACATGTAT | |
| | GGGAATTGTTCTCAATGCAACCAAAGCAGATGCCCGATTAGCG | |
| | GATGAAATTTCAGGCTAA | |
| hfsA | ATGATGCAAGACAATCAAGCCACCCAAATGCCTGTCCGGCGCG | 10 |
| | AACAACGCGGTGTGAATTTACGCGTTCGTCCACGTTTTGGCGTG | |
| | GGCGATATATTCTTGCAATTGTGGCGATCACTTTGGATTATGAT | |
| | ATTGGTGTTTCTGCCTATCGTGATACTGGGTGTTCTGTTTGCTAT | |
| | GACCATGCCCAAAACATATACGGCATTCGGCACAATGCAGGTC | |
| | ACGTTGGATAAGCAATATATTTATGACCCGCTTGTCGGAGATG | |
| | CTGGACGTGGTGTTTCTATTGAAACTGAGGCAATTGTTAGTGCG | |
| | GAAGCTGAGAAAGCCAATTCCCCTATATTAGCGCGTCGCGTTA | |
| | TGGATCAGATGGGGATTGGACACATCTATCCTAAAATTGCCCA | |
| | AGAGATAGCAGAAACGAATGACCCTGACAAAATTCGAAAATT | |
| | GGAAGCCTCTGCATATGATGCTTTGAACCAAAATTTTGGTGCAT | |
| | CTCACGGGGTGAAATCACCCTTGCTTACTTTTGTCTTTAAGCAT | |
| | GAAGATCCAATTGTTGCCGCCGAGGTTGTCAATAAATTTCTGG | |
| | AACAATTTGTCGACTATAGAGAAATTCAAAGTGATCAAGATGA | |
| | TATTGCGGCTGTGGCTGGTCAAAGATATCTTGTGGGTGAGGCCT | |
| | TAAGTGAAGCCGAATCATTGCTACGCGACTTCCTTGTTGAAAAT | |
| | GAAATTGGTGAATTTGATACTGATCGATTGTCTGTTGGCACGAC | |
| | ATTGGTGAATTTACGCAATGAACTTTTGACCGTTGAGGCATTGG | |
| | TAAAAGAAGGTGAAGGTCGTTTGAGTGGCTTGCGTGCTATGCT | |
| | GCCAACTACGCCAGAAACGATTGCGCTAGAAGTTGAGACAAAT | |
| | GCATCACAACGTGTGCTGGACCTCCAGTTAGAGCGTGAGCAAT | |
| | TATTGCTAAGATATTTGCCTGATAGCCGCGCTGTTTTAGAAATA | |
| | GATGCGCGTATAGCGAGTATGAAATCACTGTTGGAGAGTGATG | |
| | ATGGCGGTGTTCGTCGAACTGGTCCAAACCCAGCGTATCAAGA | |
| | ATTAATCTCTACAATCACGAATGTTCAATCTGATTTGAATGCGG | |
| | CAACGTCTCGCGCGGCGGAGTTAAAACGTCAGGTCAATGAAGT | |
| | GTCTCAGCGACAGAAAGAACTCGTTTTGTTGCAACCAGAATAT | |
| | AAGAAACTTCGCCGAGAGCGTGACGTGTTGGAAAGAGCGATG | |
| | GTTGAATTGGCAACGAGAGAGCAATCGAAAAAAGCAGAAGTT | |
| | CAGATTTCGGAAGGTTCAAGCGGGAGTGTTGTAATCGTCGATA | |
| | GGGCATTACCACCATCTAAAGGTTCGAGTATGAAGATACCGAT | |
| | TGTTTTGGCATCAGGGATGTTTGCTGGATTTACTGCATTAATCG | |
| | CGGGCTTACTTGTTGCATTTAGTCGTAAAAGTCTCTCAACGCGG | |
| | CGTTCAACTGAAAAGACAATAGGGTTGCCTGTCATTGGTGTGA | |
| | CATCGAAGCAATAA | |
| hfsE | ATGATGAAAAATAATGCTTCTATCCTGGCTCTGGCGGTTCTGTC | 42 |
| codon- | CATCAGCGCATGCTCCTCTCCGCAACCGGGTCCGTCTGCACCGA | |
| optimized | CCTTTCAGTCTACTACTTTCAGCAAATGGTCTCAGAACGATGCG | |
| | GCATACCGCTTTTACCCGGGCGATAAGCTGAACATTACCTTCCG | |
| | TCAGGCACCGGAGCTGGACCGTGAAGTGGTCATCGCACCTGAC | |
| | GGTCGTATTTCTCTGCCACTGATGGATCCGGTAGTGGTGGCAGA | |
| | TCTGTCCGCCTTCGAACTGCAGAAAATCCTGGAACGTATCTACG | |
| | CGCGCGAACTGGTAGATCCGAGCCTGACTGTTACCCCAGTGGA | |
| | ATTCGCCTCCCAGCAGATCTTTGTCGGTGGCGAGGTAAACAAC | |
| | CCGGGCGTTTTCCCTCTGCCAGGTCAGATCGATCCGCTGCAGGC | |
| | TATCGTGCTGGCAGGTGGCTGGAACGACAACAGCAAACCGGAA | |
| | CAGGTCATTATCCTGCGTCGCGATCGCAACGGTCAGATTATGA | |
| | CTCGCGTTGTGGACGTGAAAAACGCGCTGCGTGACCCGTCTAA | |
| | CCTGGATATCGGTCCGCTGAAACGTTTCGATGTTGTGTTCGTAT | |
| | CTCGCAGCCGCATCGCTAACGAAAATAAATTCATCCAGCAATA | |
| | TGTGCTGTCCGCGCTGCCTATCGACTTCTCTTTCTTCTACAACCT | |
| | GAAGGACAACGCGTTCTAA | |
| hfsF | ATGATGCAGGACAATCAAGCAACCCAGATGCCGGTACGCCGCG | 43 |
| codon- | AACAGCGCGGCGTTAACCTGCGTGTACGTCCGCGCTTTGGCGT | |
| optimized | GGGTGATATCTTTCTGCAACTGTGGCGCTCCCTGTGGATTATGA | |
| | TCCTGGTTTTCCTGCCGATCGTGATCCTGGGCGTGCTGTTCGCT | |
| | ATGACTATGCCGAAAACCTACACCGCGTTCGGCACCATGCAGG | |
| | TTACCCTGGATAAACAGTACATTTATGATCCGCTGGTAGGTGAC | |
| | GCGGGTCGTGGCGTCTCTATTGAAACCGAAGCAATTGTTTCCGC | |
| | TGAGGCGGAGAAAGCGAACTCTCCGATTCTGGCACGTCGCGTT | |
| | ATGGATCAGATGGGTATCGGTCACATCTACCCTAAGATCGCTC | |
| | AGGAAATTGCAGAAACCAACGACCCGGACAAAATCCGTAAAC | |
| | TGGAAGCTTCCGCTTACGACGCGCTGAACCAGAATTTCGGCGC | |
| | GTCCCACGGCGTGAAATCTCCGCTGCTGACCTTCGTCTTCAAAC | |
| | ACGAAGACCCGATCGTTGCGGCTGAGGTTGTGAACAAATTCCT | |
| | GGAACAATTCGTTGATTACCGCGAGATCCAATCCGACCAAGAT | |
| | GACATCGCGGCTGTTGCCGGTCAGCGTTACCTGGTTGGCGAAG | |
| | CTCTGAGCGAAGCTGAATCTCTGCTGCGTGATTTCCTGGTTGAG | |
| | AACGAAATTGGTGAGTTTGACACCGACCGTCTGTCTGTTGGTAC | |
| | CACTCTGGTGAACCTGCGCAACGAACTGCTGACTGTTGAAGCC | |
| | CTGGTAAAGGAAGGTGAAGGTCGTCTGTCCGGTCTGCGCGCCA | |
| | TGCTGCCGACGACTCCGGAAACTATTGCACTGGAAGTCGAGAC | |
| | CAATGCGTCTCAGCGTGTGCTGGATCTGCAGCTGGAACGTGAA | |
| | CAGCTGCTGCTGCGTTATCTGCCAGATTCCCGCGCGGTACTGGA | |
| | AATTGACGCACGTATCGCATCCATGAAAAGCCTGCTGGAGAGC | |
| | GACGATGGCGGCGTTCGTCGTACTGGCCCAAACCCAGCATACC | |
| | AGGAACTGATCAGCACTATCACGAACGTACAGTCCGATCTGAA | |
| | TGCTGCGACGTCCCGTGCGGCAGAACTGAAACGTCAGGTTAAC | |
| | GAGGTGTCCCAGCGCCAGAAGGAACTGGTACTGCTGCAGCCAG | |
| | AATACAAAAAACTGCGCCGTGAACGTGATGTGCTGGAACGCGC | |
| | TATGGTTGAACTGGCTACCCGTGAGCAGAGCAAAAAAGCGGAA | |
| | GTTCAGATCTCCGAAGGTTCCAGCGGTTCCGTAGTTATCGTGGA | |
| | TCGTGCTCTGCCGCCGTCTAAAGGTAGCTCTATGAAAATTCCGA | |
| | TCGTCCTGGCGTCTGGCATGTTTGCTGGTTTCACCGCGCTGATT | |
| | GCTGGTCTGCTGGTTGCTTTCAGCCGTAAATCCCTGTCTACCCG | |
| | CCGCTCTACTGAAAAGACTATCGGTCTGCCGGTGATCGGCGTT | |
| | ACCTCCAAACAG | |
| hfsA | ATGAAAGACCTGCGTAAAGACCTGACTGAAACCTGGCGTGTAG | 44 |
| codon- | CAACGCGTGCACCTGTTGATAACGGTGGCCGTACGATCATGTT | |
| optimized | CATGAGCGCCATGGCTGGCGAAGGTACCTCTTCTGTGGCTGCG | |
| | AGCTTTGCTATGCTGGCGGCTCAGCGTGCTCGTAAAGGTGTCTG | |
| | GCTGATCGATCTGAACCTGATGGACGGCCGCCTGTTTAATGCCT | |
| | TCGACCGTGGCGGTGACTTTGTGGACAGCTTTGGTCAGGTGGG | |
| | TCCAGCGCACAACGCAGAAATGTCCGGCGCGTCCTTCTTCAGC | |
| | ATCTCTCCTCCGCCGCCGCCGCCGCCTCCGGGTAAAAAAAACG | |
| | ACGCCGGCCTGTTCGTAATGCACCGTGTTGCGGACACGAAACT | |
| | GCTGGTGTCTCGTTTCCGCAAAGAGCGTCTGGAAAGCAGCGCA | |
| | CGTGTCCGTGTAAAAACCGGCGCGGACTATTGGAAAGCAGTTC | |
| | GTGAGATCGCTGACTGGGTTATCATCGATGCGCCGGCTCTGGA | |
| | GACCTCTTCTGCGGGTCTGGCAATTTGTTCTCAGATGGACGCGA | |
| | CCGCGCTGGTTGTGCGTGCGGACAAAACGCCAGCTACCCAGGT | |
| | ATCCAAGCTGGGCCAAGAGATCGAAGGTCACGGTGGTACCTGC | |
| | ATGGGCATCGTACTGAACGCAACCAAAGCGGATGCACGTCTGG | |
| | CAGACGAGATTTCTGGC | |
| hfsG | ATGAACACTACCCCACAGCTGAGCGCCAACCCACCAGTACGCG | 45 |
| codon- | ATACGACTACTCCACGTGAGATCGTTGTTGACAACCCGCATTG | |
| optimized | GAAAGACAAGGCCAACCAGAAACTGTCCATTCTGGTTCCGTCC | |
| | TACAAAGACGACCCGGAAGCACTGCTGGCTAGCCTGTCTAAAT | |
| | GCAAAAACGTGGAAGACATCGAATTCATCCTGTACGATGATGG | |
| | CGGTGGCGACCGTACGCTGATCGAAAAAATTAGCGCGCACGCT | |
| | GCAGAGGCTTCTTTCCCAGTTCGCATCATCTCTGCGCAACATAA | |
| | CATTGGTCGTGCGGGCGCTCGTAACCGTCTGCTGAACTACGCTC | |
| | GCTGCAAATGGGTACTGCTGCTGGACGCTGACATGCTGCCGGA | |
| | CAATGAAGACTTCCTGACCAACTACATCCAGGAAATCTCCGTT | |
| | CACCCTGATCCTAAACTGATCGTGGGCGGTTTTTCCCTGAAACA | |
| | GGCATCCACTGCTCCGGAATACGCCCTGCACCGTTGGCAGGCA | |
| | GAAAAAAGCGAATGCATGTCTTCCAAACTGCGCAACACCGAAC | |
| | CGGGCCGCTACGTCTTCACTTCCAACGTGCTGGCGCACGCAGA | |
| | TCTGTTCGAAGAGGTACCGTTCGACGAAACGTTTAGCGGTTGG | |
| | GGTTGGGAAGACGTAGACTGGGGCCTGCGCATCGCAAAGACTT | |
| | ACCTGGTTCTGCATATCGACAACACTGCTACCCACCTGGGCCTG | |
| | GATACCGACCGTGATCTGATGAAAAAGTACGGCAAGTCTGGCG | |
| | CGAACTTCAAAAAAGCAATCGAAAAGCACCCAGATGCACTGA | |
| | AAAGCACGAGCCTGTATAAGGTTGCAAACAAGCTGGCACCGAT | |
| | CCCGTTTAAACCTGTCATCAAATCTATCACGGGCAGCATCGCG | |
| | ACTGCCCACTTCTTCCCGATCAAAGTCCGTGGCCTGGCGCTGAA | |
| | ACTGTGGCGCGCGACGATCTACGCTGAAGCACTGCACGAC | |
| hfsH | ATGAACACCACCCCACAACTGTCCGCGAACCCGCCTGTACGTG | 46 |
| codon- | ATACCACTACTCCGCGTGAAATCGTAGTCGACAATCCGCACTG | |
| optimized | GAAAGACAAGGCCAACCAAAAACTGTCCATTCTGGTTCCATCC | |
| | TACAAAGATGATCCGGAAGCACTGCTGGCTTCTCTGTCCAAAT | |
| | GTAAGAACGTGGAGGACATCGAGTTCATTCTGTACGACGATGG | |
| | CGGCGGCGACCGTACTCTGATCGAAAAAATCTCTGCGCACGCC | |
| | GCAGAGGCCTCTTTCCCGGTGCGCATTATTTCCGCGCAGCATAA | |
| | CATTGGTCGTGCTGGTGCGCGCAACCGTCTGCTGAACTATGCGC | |
| | GCTGTAAGTGGGTACTGCTGCTGGATGCGGACATGCTGCCTGA | |
| | CAACGAAGACTTTCTGACCAATTACATCCAGGAGATCTCTGTG | |
| | CACCCGGATCCAAAACTGATCGTGGGCGGTTTCTCTCTGAAAC | |
| | AGGCTTCCACTGCACCAGAATACGCACTGCATCGTTGGCAGGC | |
| | CGAGAAATCTGAATGCATGTCTTCTAAGCTGCGCAACACTGAG | |
| | CCGGGCCGTTACGTTTTTACTAGCAACGTCCTGGCACATGCTGA | |
| | CCTGTTTGAAGAGGTGCCGTTCGACGAAACTTTCTCTGGTTGGG | |
| | GTTGGGAAGACGTTGATTGGGGTCTGCGTATCGCGAAGACCTA | |
| | CCTGGTACTGCACATTGACAACACTGCTACGCACCTGGGTCTG | |
| | GACACTGATCGTGATCTGATGAAAAAATATGGCAAGTCTGGCG | |
| | CGAACTTTAAAAAAGCCATCGAGAAACACCCGGACGCCCTGAA | |
| | GTCCACTAGCCTGTACAAGGTGGCAAACAAACTGGCTCCGATT | |
| | CCGTTCAAACCGGTGATTAAATCCATCACCGGTAGCATCGCGA | |
| | CCGCTCACTTCTTCCCGATTAAGGTGCGCGGCCTGGCACTGAAA | |
| | CTGTGGCGTGCGACGATCTATGCCGAAGCTCTGCACGAT | |
| hfsL | ATGAATACTACCCCACAGCTGTCTGCAAACCCACCTGTGCGCG | 47 |
| codon- | ATACTACCACCCCGCGTGAGATCGTGGTGGATAATCCACACTG | |
| optimized | GAAAGATAAGGCGAACCAGAAACTGAGCATCCTGGTGCCGTCT | |
| | TATAAAGATGACCCAGAAGCACTGCTGGCCAGCCTGTCTAAGT | |
| | GCAAAAATGTCGAAGACATCGAATTCATTCTGTATGATGACGG | |
| | CGGTGGTGACCGTACCCTGATTGAAAAAATCTCTGCGCACGCG | |
| | GCGGAAGCCTCCTTCCCGGTTCGCATTATCTCCGCGCAACACAA | |
| | TATCGGCCGCGCTGGTGCCCGTAACCGTCTGCTGAACTATGCAC | |
| | GTTGTAAATGGGTCCTGCTGCTGGATGCGGATATGCTGCCGGA | |
| | CAACGAAGATTTCCTGACGAACTACATTCAGGAAATTTCCGTG | |
| | CATCCAGATCCAAAGCTGATTGTAGGTGGCTTCAGCCTGAAAC | |
| | AGGCTTCTACCGCGCCGGAATATGCACTGCATCGTTGGCAGGC | |
| | GGAAAAAAGCGAATGCATGTCTTCCAAGCTGCGTAACACGGAA | |
| | CCGGGTCGTTACGTTTTCACTTCCAACGTTCTGGCACACGCCGA | |
| | TCTGTTCGAGGAGGTGCCATTCGATGAAACCTTCAGCGGCTGG | |
| | GGTTGGGAAGATGTTGACTGGGGTCTGCGTATTGCGAAAACCT | |
| | ACCTGGTTCTGCACATTGACAACACCGCAACTCACCTGGGCCT | |
| | GGACACCGATCGCGACCTGATGAAAAAGTACGGCAAATCTGGT | |
| | GCGAACTTCAAAAAAGCGATCGAAAAGCATCCGGACGCGCTG | |
| | AAATCTACGTCTCTGTACAAAGTCGCTAACAAACTGGCGCCGA | |
| | TTCCGTTTAAACCGGTCATCAAATCCATCACCGGTTCTATTGCA | |
| | ACGGCTCACTTCTTCCCGATTAAAGTTCGTGGCCTGGCTCTGAA | |
| | ACTGTGGCGTGCCACTATCTATGCGGAAGCACTGCACGAC | |
| hfsC | CTGTTCAACAACGATACCTGTCTGTCTAAATGGCACAACTCTCC | 48 |
| codon- | GCACTCCCTGAACGGCCCACTGTACTGGATTGAGCGTGCTCTG | |
| optimized | GTTTTCTTCATCGTGCTGACTTACAGCGGCCTGTGGATTGCAAT | |
| | CCTGCTGGGTCGTGCAGCGGAAACGCAGTCTATGATCGCGCCA | |
| | GACCCGGGTCCGATTGCTCGTGCCAGCTGGTTCCCGGCGTATCT | |
| | GGCACTGATCGGTCTGCTGGGCCTGAATATTAAAAAACTGGGT | |
| | TCTGCGTCCCTGAAATTCTGGCCAATCATCCTGCTGCTGGGTCT | |
| | GGCGGTTGCGAGCGCGAACTGGTCTCTGGATCCTTCTCTGACCC | |
| | AGCGTCGTGCTATTGCCCTGAGCTTCTCCTTCATGTTCGGTTGC | |
| | TATCTGGCTATCCGTGCTCCGCTGGTAGACACCCTGCGTATTAT | |
| | CGGCTGGGCCTGGCTGACGATCTGCATTCTGAACTTTCTGCTGA | |
| | TCTTCGGCGCTCCTCACCTGGGTGTTCATTCCGAACTGCACGTT | |
| | GGCGCGTGGCGCGGCTTCATGACTGAGAAAAACCATCTGGGCG | |
| | GTGAAATGGCCCGTGCTAACCTGATCTTCCTGGCTCTGGTCTAT | |
| | TTCGACCGTAAGACCCCGGCAGGCCAGAACAAAAAAGCATGGT | |
| | GGCTGGGCCTGGCGCTGACCTGGATGCTGATCCTGGGTTCCAC | |
| | CTCCAAAACGGCCCTGATCGCGACGCTGATCCCGTATCTGGGTT | |
| | TTCTGTTCTACAGCATCGCAATCCGTACTCCGATCCTGGGTCTG | |
| | CTGGCGTTCTGGGGTGGCCTGTCTCTGGCTGGCATCGGTTACGC | |
| | AATCATCAGCATCAGCCCAGAAACCGTGGTGGCACTGATCGGC | |
| | AAGGACCTGACTTTCACTGGTCGCACCGGTATCTGGGCGATCG | |
| | TTATCGACCTGATCAATCAGCAGAAATGGACGGGCTATGGCTA | |
| | CGGTGCTTTCTGGGTGACCCCGGATGGCCCGGTAGCTTTCATTG | |
| | TCAACACCCTGGAATGGAACGTGCCAACCGCTCACAACGGTTG | |
| | GCTGGAAGTGGGCCTGGCGATTGGCTATCCGGGCCTGATTCTG | |
| | ATCATCTGCATCTCTCTGTTCGCTCTGGGTAAAGCCGCCTACCT | |
| | GGCTACCGGCAAACACGGTCCGTTCGTTTTCCTGATGCTGTTCC | |
| | AGATCATTCTGTTCTCCCTGTCCGAAAGCATCCTGATGCAGCAG | |
| | AACAGCCACGCATCTTCTCTGTTCTACTTCTTCACCGCTTACGC | |
| | TTTTATCGCACGTAAAGTGGCGACCGACTCCCAGACTCCGCTGC | |
| | TGTCTGCGCCGCACTGGGCACTGCCTCCGCGTACTACCAAAAA | |
| | ACGTAGC | |
| hfsB | ATGAAAGACCTGCGTAAAGACCTGACTGAAACTTGGCGTGTGG | 49 |
| codon- | CGACTCGTGCACCGGTTGACAATGGTGGTCGTACCATCATGTTC | |
| optimized | ATGTCCGCCATGGCTGGCGAAGGTACCTCCAGCGTGGCAGCGT | |
| | CCTTTGCGATGCTGGCTGCTCAGCGTGCGCGCAAAGGTGTTTGG | |
| | CTGATCGATCTGAACCTGATGGACGGTCGTCTGTTCAACGCGTT | |
| | TGACCGCGGTGGTGATTTCGTAGACTCTTTCGGTCAGGTCGGCC | |
| | CGGCTCATAACGCTGAGATGAGCGGTGCTTCTTTCTTTTCCATT | |
| | TCTCCGCCACCACCGCCGCCACCGCCAGGCAAAAAGAACGATG | |
| | CAGGCCTGTTCGTGATGCACCGTGTTGCGGACACCAAACTGCT | |
| | GGTGAGCCGCTTCCGTAAAGAACGTCTGGAAAGCAGCGCGCGC | |
| | GTCCGTGTGAAAACCGGTGCGGACTACTGGAAGGCTGTTCGTG | |
| | AAATCGCTGACTGGGTGATCATTGATGCCCCGGCTCTGGAAAC | |
| | CTCCTCTGCAGGTCTGGCTATCTGCTCCCAGATGGATGCTACCG | |
| | CTCTGGTTGTGCGTGCAGATAAAACCCCAGCGACCCAGGTAAG | |
| | CAAACTGGGCCAGGAGATCGAAGGTCACGGTGGCACTTGCATG | |
| | GGCATTGTGCTGAACGCGACCAAAGCGGATGCACGTCTGGCAG | |
| | ATGAAATCAGCGGC | |
| hfsD | ATGATTTGGCGCCACCTGTTCGGCTACCTGCCTGTAAACGTTAT | 50 |
| codon- | CCAGGGCCTGGTGTCCTTCGGTGCGGTATACGCTTTCACTCGCC | |
| optimized | TGCTGGGCGATGACGGCTACGGCAGCTATGCTCTGGTTCTGAC | |
| | CATCATGTCCGCAAGCCACACCACCACGCTGACTTGGACTGAA | |
| | GCAGCAGCGTATCGTTTCGCGGGTGAAGCGCAGAGCAAAGGTG | |
| | GCATGAATGACCACATCCGTACGAGCATCTACCTGGCGCTGTT | |
| | CAGCCTGATCCCAGCTCTGCTGATTGTGGCATGCGGCTGGATG | |
| | GCATCCGAAAACAACCCGAACATGCAGGCGGCAATCATCTGGC | |
| | TGGCTCTGTCTATGCCGTGCCTGTCTATTATCCAAATGAGCCTG | |
| | GAAATTCACAAGGCGCGTCAGCAGGTTTCCCGTTTCGCAAAGG | |
| | TCTCTATCGCCCACGCTCTGACTGGTTTCTGTGGTGGCCTGTAT | |
| | TTCGCATCTCAGACTGATGCAGGTGCGGCAGCGCCTTTTATGGG | |
| | CCTGGCTCTGTCCGGTGTAATCTTTGCAAGCGTGCAGGGCCTGT | |
| | TCCTGTGGAAAGAATCTAAAGACGGTTCTTTCCAGATGGTACG | |
| | TGCGAAACGTTACTTTGCTTACGGCATGCCGCTGGCTCTGGCTC | |
| | TGCTGCTGGAAATTGCACTGTCTGCGTCCGATCGTTTTCTGATC | |
| | GCCTACTTCATCGATAACGCAGCTGTTGGTGCGTATGCTGCCGG | |
| | TTATGGCGTGAGCGATCAGTCCATCCGTCTGCTGTGCATGTGGG | |
| | GTGCGATGGCTGGTGCGCCTCTGCTGATGGAATCTTACGAGAA | |
| | ACACGGTCTGAACGGCATCGAAGAACCGGGTAAAGCAATGATC | |
| | CGTATGCTGATGCTGATCGCGTTTCCGGCCGCAACCGGTCTGGC | |
| | GATGGTGGCCGAACCGCTGGCACAGTTTATGATCGGTGAGGAG | |
| | CTGCGTGATCAGGCGAAACACACCATTCCGTGGATTGCACTGG | |
| | CCGGCCTGATGAACGGTCTGGTGATCTATTACTTCTCCGAATCC | |
| | TTCCAGCTGGCACGTAAAACCGCCCTGCGTGCATCTCTGATGCT | |
| | GATCCCGGCCATCCTGAACGTAATCCTGAACATCATCCTGCTGC | |
| | CTAAAATGGGCCTGATGGGTGCTGTATATGCGACTGTTATTTGT | |
| | TACGGCGTTGCGCTGATTATCATCATGGGTGTAGGTCGTCGTTT | |
| | CATCCCGCTGCCGGTTCCGATGAAAGACATTGTGCTGATCGCC | |
| | ATTGCGTGTGCGGGTATGGCGAGCATCGTTTACATCCTGCCGCA | |
| | AATCGGTGGCTTCCCGGAACTGATGCTGAAGGCCATCGTAGGC | |
| | GGTATCATCTATGGTGTACTGGCAATCGTACTGAACGCAGCTG | |
| | GTGCCAAAGATCTGATTAAAGCGCTGAAAGACCGTAAAAACGC | |
| | TACCCAG | |
| hfsJ | ATGAGCTCTGGTAAAAAGATCTACGACATGCTGGAACACCTGA | 51 |
| codon- | CTGTACCTGAAACCGAAGCCAACGTTGACGCACTGCTGAATGG | |
| optimized | TCTGCCGGACGCTACTTCTCCGCAGGTTATCAGCTTCGTTAACG | |
| | CTCACGCAGTAAACCTGATGGTGAAAGACGAAGGTCTGTTCAA | |
| | AGCGCTGATCGGTTCTGACATCCTGCTGCGCGACGGCTCTGGC | |
| | ATGAAAATCCTGATGAAGTGGCTGAACCAAAACCCGGGTGCAA | |
| | ACCTGAACGGCACCGATCTGATTCCGCGTATTATCGAGAAGTT | |
| | CGATGGTATGAAAGTGGCGGTTTTCGGTACCCAAGAACCATGG | |
| | CTGTCTAAAGGTTGCGACGTAATTGAAACCCGTGGCGGCACCA | |
| | TCGTGTCCCGTCTGAACGGTTTCCAAGATGAAGCCGCTTACATT | |
| | GAGGCGATCGAAACCTCCAAACCGGACCTGGTAATCCTGGCTA | |
| | TGGGTATGCCGAAACAGGAAATGACGTCTATGGCACTGCGTGC | |
| | GGCCGCCTCTTGGCCAACCACCATTGTCAACGGTGGTGCAATC | |
| | ATCGATTTCCTGGCGGAACGCGTAAACCGTGCTCCGGAAACTT | |
| | GGCGTAAACTGGGTATGGAATGGCTGTACCGTCTGATTCAAGA | |
| | GCCGAAACGCCTGTTCGGTCGCTACGTAGTCGGCAACGTTATCT | |
| | TCCTGACGCGTGGTCTGATTCTGTGCGTTACTCAAGCGAACCCG | |
| | AAAATCACC | |

In some cases, the engineered cell may comprise one or more exogenous promoter sequences for the one or more exogenous nucleic acid sequences that regulates production of the bioadhesive (e.g., holdfast). Table 2 shows exemplary promoter sequences for the one or more exogenous nucleic acids. In some cases, the engineered cell may comprise an exogenous nucleic acid sequence having at least about 70%, at least about 75%, at least about 80%, at least about 85%, at least about 90%, or at least about 95% sequence identity to SEQ ID NO: 1 or 51 with a promoter sequence SEQ ID NO: 11 or 21. In some cases, the engineered cell may comprise an exogenous nucleic acid sequence having at least about 70%, at least about 75%, at least about 80%, at least about 85%, at least about 90%, or at least about 95% sequence identity to SEQ ID NO: 2 or 53 with a promoter sequence SEQ ID NO: 12 or 22. In some cases, the engineered cell may comprise an exogenous nucleic acid sequence having at least about 70%, at least about 75%, at least about 80%, at least about 85%, at least about 90%, or at least about 95% sequence identity to SEQ ID NO: 3 or 55 with a promoter sequence SEQ ID NO: 13 or 23. In some cases, the engineered cell may comprise an exogenous nucleic acid sequence having at least about 70%, at least about 75%, at least about 80%, at least about 85%, at least about 90%, or at least about 95% sequence identity to SEQ ID NO: 4 or 59 with a promoter sequence SEQ ID NO: 14 or 24. In some cases, the engineered cell may comprise an exogenous nucleic acid sequence having at least about 70%, at least about 75%, at least about 80%, at least about 85%, at least about 90%, or at least about 95% sequence identity to SEQ ID NO: 5 or 54 with a promoter sequence SEQ ID NO: 15 or 25. In some cases, the engineered cell may comprise an exogenous nucleic acid sequence having at least about 70%, at least about 75%, at least about 80%, at least about 85%, at least about 90%, or at least about 95% sequence identity to SEQ ID NO: 6 or 51 with a promoter sequence SEQ ID NO: 16 or 26. In some cases, the engineered cell may comprise an exogenous nucleic acid sequence having at least about 70%, at least about 75%, at least about 80%, at least about 85%, at least about 90%, or at least about 95% sequence identity to SEQ ID NO: 7 or 56 with a promoter sequence SEQ ID NO: 17 or 27. In some cases, the engineered cell may comprise an exogenous nucleic acid sequence having at least about 70%, at least about 75%, at least about 80%, at least about 85%, at least about 90%, or at least about 95% sequence identity to SEQ ID NO: 8 or 58 with a promoter sequence SEQ ID NO: 18 or 28. In some cases, the engineered cell may comprise an exogenous nucleic acid sequence having at least about 70%, at least about 75%, at least about 80%, at least about 85%, at least about 90%, or at least about 95% sequence identity to SEQ ID NO: 9 or 57 with a promoter sequence SEQ ID NO: 19 or 29. In some cases, the engineered cell may comprise an exogenous nucleic acid sequence having at least about 70%, at least about 75%, at least about 80%, at least about 85%, at least about 90%, or at least about 95% sequence identity to SEQ ID NO: 10 or 52 with a promoter sequence SEQ ID NO: 20 or 30.

TABLE 2
Nucleic Acid Protomer Region Sequences

| | Original DNA sequence | | Modified DNA sequence | |
|---|---|---|---|---|
| Gene or | othe promoter region | SEQ ID | of the promoter region | SEQ ID |
| region | of the gene | NO: | of the gene | NO: |
| hfsE | CTATAAGCAATGAAGCA | 11 | GATCCCGCGAAATTAAT | 21 |
| | TTACTCATACTAGCCTT | | ACGACTCACTATAGGGG | |
| | CTTTTGAAGTGTTCATG | | AATTGTGAGCGGATAAC | |
| | CTAGGTTGTATTGCACA | | AATTCCCCTCTAGAAATA | |
| | AGGTAAATGGAGACACT | | ATTTTGTTTAACTTTAAG | |
| | CTGTGTTCTTCGTGA | | AAGGAGATATACC | |
| hfsG | ATATATGGAAAAAGCAT | 12 | TCTGGTTTGATCTATACG | 22 |
| | CATTCTGGTTTGATCTAT | | TCATCCTGATGACTGCTC | |
| | ACGTCATCCTGATGACT | | CCTGCCTTTTGGGCGACT | |
| | GCTCCCTGCCTTTTGGG | | CAAAAACACAGCGCTAG | |
| | CGACTCAAAAACACAGC | | TTTGTTTAACTTTAAGAA | |
| | GCTAGGCATCCAAT | | GGAGATATACC | |
| hfsL | CCACAACTGGGATCGTC | 13 | CAATAAAGGTTAGAGGT | 23 |
| | GAAGAATTGTGCCAACT | | CTCGCCCTAAAATTATGG | |
| | GTCTAAAGCAGCAGGCG | | CGTGCAACCATATATGC | |
| | CAACGCTTTCAACGCCA | | GGAGGCTCTTCATGATTG | |
| | ACTGAGGCAGCAAGAA | | ATTTGTTTAACTTTAAGA | |
| | GCTACGGCCTAATCTC | | AGGAGATATACC | |
| hfsJ | GATGGTCGAACTTATGC | 14 | TTATTCTGCAATTTTATG | 24 |
| | TTCCGCTTGGTTTGGTTG | | GGGATATTCCCGATAAG | |
| | AAGTCTCAAGAACGGGT | | AAAAAGCGCAAAAAAGT | |
| | GTATTATCGCTTCAGCG | | TATCAAAAGCGCAACTT | |
| | CGGCCCTCATGCGGGAG | | AATTTGTTTAACTTTAAG | |
| | CTGGTCGGTAGATT | | AAGGAGATATACC | |
| hfsH | TCCATAACTGGTAGCAT | 15 | TTGGAAATGTCATTTTTC | 25 |
| | TGCGACAGCGCACTTTT | | TCACGCGTGGTTTGATAT | |
| | TTCCAATAAAGGTTAGA | | TATGTGTGACCCAAGCT | |
| | GGTCTCGCCCTAAAATT | | AATCCCAAAATAACTTA | |
| | ATGGCGTGCAACCATAT | | ATTTGTTTAACTTTAAGA | |
| | ATGCGGAGGCTCTTC | | AGGAGATATACC | |
| hfsF | TTCAAGTCGGCAGCTAA | 16 | ACTCACAAACACCTCTCT | 26 |
| | GAAAGATTGTATTTACA | | TAAGCGCCCCGCATTGG | |
| | AGTCTCTTTCTATAGCC | | GCGCTACCACCGCGTAC | |
| | AGTCTAACGAGACACTT | | TACGAAGAAACGCAGCT | |
| | AGAATAGACAGTTTTAG | | AATTTGTTTAACTTTAAG | |
| | GAACAAATATAACAG | | AAGGAGATATACC | |
| hfsC | TTAAGGGCTGAGATTGC | 17 | AGCAATACGTACTATCA | 27 |
| | ACCCTTGTAAGAAGGTG | | GCATTGCCAATTGATTTT | |
| | CAAAACAATTGAATGAT | | TCATTCTTCTACAACCTA | |
| | CATGCTGTATTAACCTC | | AAAGACAACGCCTTCTA | |
| | AACGCATCTTTCCATAT | | ATTTGTTTAACTTTAAGA | |
| | AACGGAGCAGAAAAG | | AGGAGATATACC | |
| hfsD | CATCATATTCTGATTCT | 18 | GCACATGTATGGGAATT | 28 |
| | GCTTTATCTAAACAACT | | GTTCTCAATGCAACCAA | |
| | TGTTCATTTGTCGCTTGA | | AGCAGATGCCCGATTAG | |
| | TTTGCCTTAATATTTCAT | | CGGATGAAATTTCAGGC | |
| | TATCCAAAACGGATGCA | | TAATTTGTTTAACTTTAA | |
| | TTAAGAAAGCACC | | GAAGGAGATATACC | |
| hfsB | TTGTTATTTAATTAAAA | 19 | GTCTCTCAACGCGGCGTT | 29 |
| | AATATACTTTTCAAAAA | | CAACTGAAAAGACAATA | |
| | GTTAACTTAGTTTTTCTA | | GGGTTGCCTGTCATTGGT | |
| | TGTTGAAACCAATTATA | | GTGACATCGAAGCAATA | |
| | GCGGCAAAATAAGATGT | | ATTTGTTTAACTTTAAGA | |
| | AGTGAATTTGAGTG | | AGGAGATATACC | |
| hfsA | CATCATGGTGCTTTCTT | 20 | TTCCTCCTTTCAGTAATA | 30 |
| | AATGCATCCGTTTTGGA | | CGACTCACTATAGGGGA | |
| | TAATGAAATATTAAGGC | | ATTGTGAGCGGATAACA | |
| | AAATCAAGCGACAAAT | | ATTCCCCTCTAGAAATAA | |
| | GAACAAGTTGTTTAGAT | | TTTTGTTTAACTTTAAGA | |
| | AAAGCAGAATCAGAAT | | AGGAGATATACC | |

In some embodiments, the engineered cell may encode a polysaccharide deacetylase enzyme HfsH (SEQ ID NO: 31) that modulates holdfast binding properties. In some embodiments, the level of activity of HfsH correlates with adhesiveness of holdfast polysaccharides. In some embodiments, as shown in Table 3, mutations in the different part of the enzyme (SEQ ID NO: 32) are made in order to create better, more hyperactive, and more stable enzymes. In some embodiments, the sequences are modified for E. coli and yeast expression systems.

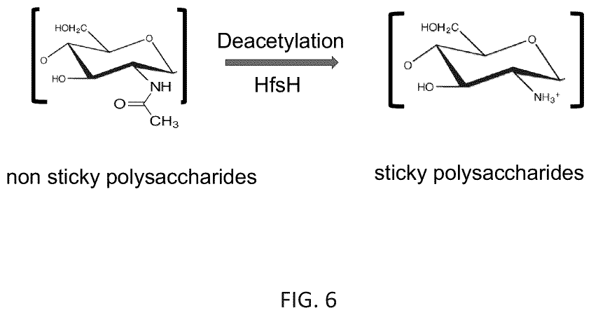

FIG. 6 shows an exemplary deacetylation of holdfast polysaccharides. In some cases, the engineered cell may produce non-sticky polysaccharides. The non-sticky polysaccharides may comprise an acetamide group. With a deacetylation enzyme (e.g., hfsH), the acetamide group may be deacetylated to form an ammonia or ammonium group. Polysaccharides comprising the ammonia or ammonium group is sticky. Therefore, with the deacetylation, the non-sticky polysaccharides may be transformed to sticky polysaccharides.

TABLE 3
HfsH protein Sequences

| | Original | SEQ | Modified | SEQ ID |
|---|---|---|---|---|
| Protein | protein sequence | ID NO: | protein sequence | NO: |
| HfsH | MIDWHYTPSRTLPAKLKR | 31 | MHHHHHHMIDWHYTPS | 32 |
| | RMTQWRHAAPVDVSNTQ | | RTLPAKLKRRMTQWRH | |
| | FHVSYTFDDFPMSAVNGA | | DAPVDVSNTQFHVSYTF | |
| | DILESHDGHAAFYACTKM | | DDFPMSAVNGADILESH | |
| | IGTHGAYGDMYDIKTML | | DGHLAFYACTKMIGTH | |
| | DLENRGHEIGAHTHSHLD | | GAYGDMYDIKTMLDLE | |
| | CAQSKRETVLNDIDANIS | | NRGHEIGAHTHSHLDCA | |
| | ALMEAGLKKRPTSFAYPY | | QSKRETVLNDIDANISA | |
| | GETLFDTKKEVFKKFDLC | | LMEAGLKKRPTSFKYPY | |
| | RGILPGINVGKVDLAQLR | | GETLFDTKKEVFKKFDL | |
| | CFELNENPATRIRAINAIE | | CRGILPGINVGKLDLAQ | |
| | EAGKTGGWVIIFTHDVSP | | LRCFELNENPATRIRAIN | |
| | QPTAYGTTTGIVEELCQLS | | AIEEAGKTGGWVIIFTH | |
| | KAAGATLSTPTEAARSYG | | DVSPQPTAYGTTTGIVE | |
| | LIS | | ELCQLSKAAGATLSTPT | |
| | | | EAARSYGLISYPYDVPD | |
| | | | YA | |

The sequences disclosed in Table 1 correspond to genes identified from bacterium that are used in the synthesis of the bioadhesive (e.g., holdfast). In some embodiments, the genus of bacterium wherein the genes are derived from include Caulobacterales bacterium. In some embodiments, the genes are derived from a species of Caulobacterales bacterium. In some embodiments, the species includes Hirschia baltica bacterium or Caulobacter crescentus bacterium. In some embodiments, the species includes Hirschia baltica bacterium. In some embodiments, the species includes Caulobacter crescentus bacterium.

Because the one or more genes are used in the synthesis of the holdfast, the genes are associated with and encode one or more proteins that regulate production of the holdfast. HfsE gene may encode production of exopolysaccharide biosynthesis polyprenyl glycosylphosphotransferase (e.g., HfsE enzyme). HfsG gene may encode production of glycosyl transferase. HfsL may encode production of glycosyl transferase (e.g., HfsG enzyme). HfsJ gene may encode production of glycosyl transferase (e.g., HfsJ enzyme). HfsH gene may encode production of polysaccharide deacetylase (e.g., HfsH enzyme). HfsA gene may encode production of lipopolysaccharide biosynthesis protein (e.g., HfsA enzyme). HfsB gene may encode production of polysaccharide autokinase-related protein (e.g., HfsB enzyme). HfsD gene may encode production of polysaccharide export protein (e.g., HfsD enzyme). HfsC gene may encode production of O-antigen polymerase (e.g., HfsC enzyme). HfsD gene may encode production of polysaccharide biosynthesis protein (e.g., HfsD enzyme). In some cases, the genes are associate with and encode one or more of glycosyl transferases, exopolysaccharide biosynthesis polyprenyl glycosylphosphotransferases, polysaccharide deacetylases, lipopolysaccharide biosynthesis proteins, polysaccharide autokinase-related proteins, O-antigen polymerases, or polysaccharide biosynthesis proteins, or any combination thereof. In some cases, the engineered cell may produce one or more of glycosyl transferases, exopolysaccharide biosynthesis polyprenyl glycosylphosphotransferases, polysaccharide deacetylases, lipopolysaccharide biosynthesis proteins, polysaccharide autokinase-related proteins, O-antigen polymerases, or polysaccharide biosynthesis proteins, or any combination thereof. In some cases, the enzymes provided herein can facilitate the production of the bioadhesive or bioadhesive component(s).

In some embodiments, the bioadhesive comprises holdfast (e.g., that is produced by the bacteria described above). In some embodiments, the holdfast comprises one or more polysaccharides. In some embodiments, the holdfast polysaccharides contain N-acetylglucosamine (GlcNAc), glucose, 3-O-methylglucose, mannose and xylose residues. In some embodiments, the adhesive subunit of holdfast is the deacetylated GlcNAc moieties. In some instances, holdfast adhesiveness is modulated by the deacetylase enzymes that removes the acetyl group leaving a positive charge on GlcNAc residue. In some instances, the positively charged residues forms covalent bonds with substrate molecules.

In some embodiments, the nucleic acid sequences may include a spacer. In some embodiments, the spacer may be incorporated into the nucleic acid sequence between each of the genes incorporated into the nucleic acid sequence. In some embodiments, the genes may be any one of the genes disclosed in Table 1. In some embodiments, the spacer may comprise a sequence having at least about 70%, at least about 75%, at least about 80%, at least about 85%, at least about 90%, or at least about 95% sequence identity to SEQ ID NOs: 33-35 as disclosed in Table 4. In some embodiments, the spacer is used to split the one or more nucleic acid sequences into two or more groups. In some embodiments, the spacer is used to split the 10 genes into two groups that are expressed in opposite directions. In some embodiments, the spacer is used between two groups of five holdfast genes to facilitate expression of a single holdfast gene. In some embodiments, the spacer enables the 10 genes to be expressed individually to make 10 holdfast proteins. In some embodiments, the one or more exogenous nucleic acid sequences comprise each of SEQ ID NOs: 1-10, and the spacer is included between SEQ ID NO. 5 and SEQ ID NO. 6. In some embodiments, the one or more exogenous nucleic sequences comprise each of SEQ ID NOs: 1-10, and the spacer is included between one or more of SEQ ID NOs: 1-10.

In some cases, the engineered cell may comprise two or more of the one or more exogenous nucleic acids in sequence. In some cases, the two or more of the exogenous nucleic acids in sequence may not comprise a promoter sequence in between two exogenous nucleic acids. In some cases, the two or more of the exogenous nucleic acids in sequence may comprise a promoter sequence in between the two or more exogenous nucleic acids. In some cases, the engineered cell may comprise a promoter sequence upstream of a first exogenous nucleic acid of the two or more of the exogenous nucleic acids in sequence. In some cases, depending on the order of the exogenous nucleic acids, the promoter sequence may be any suitable sequence disclosed in Table 2. In some cases, the engineered cell may comprise a T7 terminator with a sequence of SEQ ID NO: 37 as disclosed in Table 4. In some cases, the engineered cell may comprise a T7 promoter with a sequence of SEQ ID NO: 38 as disclosed in Table 4. In some cases, the engineered cell may comprise a kanamycin R sequence with a sequence of SEQ ID NO: 39 as disclosed in Table 4. In some cases, the engineered cell may comprise a lac operator with a sequence of SEQ ID NO: 40 as disclosed in Table 4. In some cases, the engineered cell may comprise a lacI promoter with a sequence of SEQ ID NO: 41 as disclosed in Table 4.

TABLE 4
Other Sequences

| | DNA Sequence | SEQ ID NO: |
|---|---|---|
| Spacer | GATCCCGCGAAATTAATACGACTCACTATAGGGGAA | 33 |
| | TTGTGAGCGGATAACAATTCCCCTCTAGAAATAATT | |
| | TTGTTTAACTTTAAGAAGGAGATATACC | |
| Spacer | GATCCGGCTGCTAACAAAGCCCGAAAGGAAGCTGA | 34 |
| | GTTGGCTGCTGCCACCGCTGAGCAATAACTAGCATA | |
| | ACCCCTTGGGGCCTCTAAACGGGTCTTGAGGGGTTT | |
| | TTTGATCGACGGATCGGGAGATCTCCCGATCCCCTA | |
| | TGGTTCTCAGTACAATCTGCTCTGATGCCGCATAGTT | |
| | AAGCCAGTATCTGCTCCCTGCTTGTGTGTTGGAGGTC | |
| | GCTGAGTAGTGCGCGAGCAAAATTTAAGCTACAACA | |
| | AGGGTACCCAAAAAACCCCTCAAGACCCGTTTAGAG | |
| | GCCCCAAGGGGTTATGCTAGTTATTGCTCAGCGGTG | |
| | GCAGCAGCCAACTCAGCTTCCTTTCGGGCTTTGTTAG | |
| | CAGCCGGATC | |
| Spacer | ATCGACGGATCGGGAGATCTCCCGATCCCCTATGGT | 35 |
| | TCTCAGTACAATCTGCTCTGATGCCGCATAGTTAAGC | |
| | CAGTATCTGCTCCCTGCTTGTGTCATGTTAGACGAGA | |
| | CTACGGCGTATCAATTCGGTCATAGACGAGGGACGA | |
| | ACAGTGTTGGAGGTCGCTGAGTAGTGCGCGAGCAAA | |
| | ATTTAAGCTACAACAAGGGTACC | |
| Ribosome | tttgtttaactttaagaaggaga | 36 |
| binding | | |
| site | | |
| T7 | ctagcataaccccttggggcctctaaacgggtcttgaggggttttttg | 37 |
| terminator | | |
| T7 | TAATACGACTCACTATAGGG | 38 |
| promoter | | |
| Kanamycin | ttagaaaaactcatcgagcatcaaatgaaactgcaatttattcatatcaggattatcaatac | 39 |
| R | catatttttgaaaaagccgtttctgtaatgaaggagaaaactcaccgaggcagttccata | |
| | ggatggcaagatcctggtatcggtctgcgattccgactcgtccaacatcaatacaacct | |
| | attaatttcccctcgtcaaaaataaggttatcaagtgagaaatcaccatgagtgacgact | |
| | gaatccggtgagaatggcaaaagtttatgcatttctttccagacttgttcaacaggccag | |
| | ccattacgctcgtcatcaaaatcactcgcatcaaccaaaccgttattcattcgtgattgcg | |
| | cctgagcgagacgaaatacgcgatcgctgttaaaaggacaattacaaacaggaatcg | |
| | aatgcaaccggcgcaggaacactgccagcgcatcaacaatattttcacctgaatcagg | |
| | atattcttctaatacctggaatgctgttttcccggggatcgcagtggtgagtaaccatgca | |
| | tcatcaggagtacggataaaatgcttgatggtcggaagaggcataaattccgtcagcca | |
| | gtttagtctgaccatctcatctgtaacatcattggcaacgctacctttgccatgtttcagaa | |
| | acaactctggcgcatcgggcttcccatacaatcgatagattgtcgcacctgattgcccg | |
| | acattatcgcgagcccatttatacccatataaatcagcatccatgttggaatttaategcg | |
| | gcctagagcaagacgtttcccgttgaatatggctcat | |
| Lac | ggaattgtgagcggataacaattcc | 40 |
| operator | | |
| LacI | tcactgcccgctttccagtcgggaaacctgtcgtgccagctgcattaatgaatcggcca | 41 |
| | acgcgcggggagaggcggtttgcgtattgggcgccagggtggtttttcttttcaccagt | |
| | gagacgggcaacagctgattgcccttcaccgcctggccctgagagagttgcagcaag | |
| | cggtccacgctggtttgccccagcaggcgaaaatcctgtttgatggtggttaacggcg | |
| | ggatataacatgagctgtcttcggtatcgtcgtatcccactaccgagatatccgcaccaa | |
| | cgcgcagcccggactcggtaatggcgcgcattgcgcccagcgccatctgatcgttgg | |
| | caaccagcatcgcagtgggaacgatgccctcattcagcatttgcatggtttgttgaaaac | |
| | cggacatggcactccagtcgccttcccgttccgctatcggctgaatttgattgcgagtga | |
| | gatatttatgccagccagccagacgcagacgcgccgagacagaacttaatgggcccg | |
| | ctaacagcgcgatttgctggtgacccaatgcgaccagatgctccacgcccagtcgcgt | |
| | accgtcttcatgggagaaaataatactgttgatgggtgtctggtcagagacatcaagaa | |
| | ataacgccggaacattagtgcaggcagcttccacagcaatggcatcctggtcatccag | |
| | cggatagttaatgatcagcccactgacgcgttgcgcgagaagattgtgcaccgccgct | |
| | ttacaggcttcgacgccgcttcgttctaccatcgacaccaccacgctggcacccagttg | |
| | atcggcgcgagatttaatcgccgcgacaatttgcgacggcgcgtgcagggccagact | |
| | ggaggtggcaacgccaatcagcaacgactgtttgcccgccagttgttgtgccacgcg | |
| | gttgggaatgtaattcagctccgccatcgccgcttccactttttcccgcgttttcgcagaa | |
| | acgtggctggcctggttcaccacgcgggaaacggtctgataagagacaccggcatac | |
| | tctgcgacatcgtataacgttactggtttcac | |

In some embodiments, the engineered cell is engineered from a competent cell. In some embodiments, the competent cell comprises an E. coli cell. In some embodiments, the E. coli cell comprises an E. coli BL21 (DE3) competent cell.

In some embodiments, the nucleic acid sequence may include or be attached to one or more ribosome binding sites (RBSs). A ribosome binding site (RBS) is a sequence of nucleotides in a messenger RNA (mRNA) molecule that binds to the ribosome. The RBS is located upstream of the start codon of the mRNA transcript. It is responsible for recruiting a ribosome during the initiation of translation. The RBS controls the accuracy and efficiency of the translation of mRNA. Different RBSs can affect the efficiency of fluorescent protein translation and thus the signal to noise ratio. In bacteria, translation initiation almost always requires both an RBS sequence and a start codon. In eukaryotes, ribosome recruitment is generally mediated by the 5′ cap present on eukaryotic mRNAs. In some cases, each exogenous gene disclosed herein may comprise a ribosome binding site. In some cases, the ribosome binding site may have a sequence having at least about 70%, at least about 75%, at least about 80%, at least about 85%, at least about 90%, or at least about 95% sequence identity to SEQ ID NO: 36 as disclosed in Table 4. In some cases, the engineered cell may comprise a coupling sequence (tatacc) to connect the RBS and the exogenous nucleic acid.

In some embodiments, each of the RBSs ensures each holdfast gene is translated into a single protein. This can ensure that the 10 holdfast genes (SEQ ID Nos 1-10) produce 10 holdfast proteins.

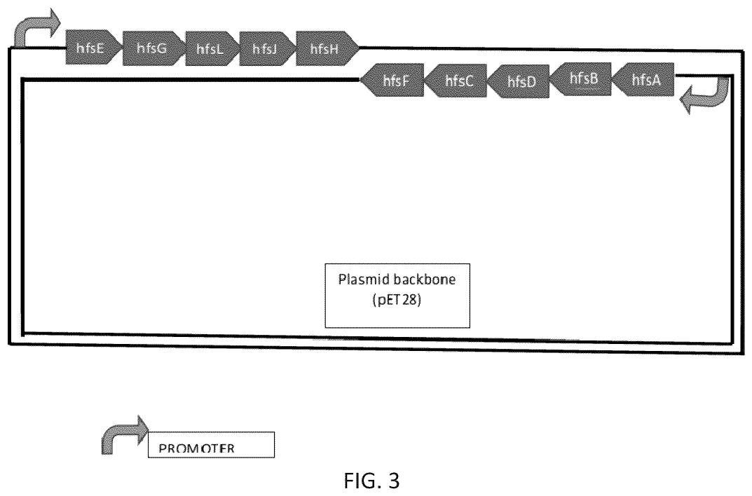

FIG. 3 shows an exemplary nucleic acid-plasmid construct. The construct comprises 10 exogenous nucleic acids (or genes) cloned to a plasmid (e.g., pET28) or derivative. The construct may comprise hfsE, hfsG, hfsL, hfsJ, and hfsH in a sense or “+” strand. The construct may further comprise hfsA, hfsB, hfsD, hfsC, and hfsF in an antisense or “−” strand. The construct may comprise a forward promoter upstream of hfsE gene. The construct may comprise a reverse promoter upstream of hfsA gene. The forward promoter and the reverse promoter may be same. The forward promoter and the reverse promoter may be different. The forward promoter and/or the reverse promoter may be a T7 promoter. The forward promoter and/the reverse promoter may be a T7 promoter disclosed in Table 2. The construct may further comprise a spacer between the hfsH gene and the hfsF gene. The construct may express all 10 genes in a BL21 strain. The construct may further comprise a RBS between two adjacent genes. In some cases, the genes on the sense strand may be any combination of the genes in any order disclosed herein. In some cases, the genes on the antisense strand may be any combination of the genes in any order disclosed herein.

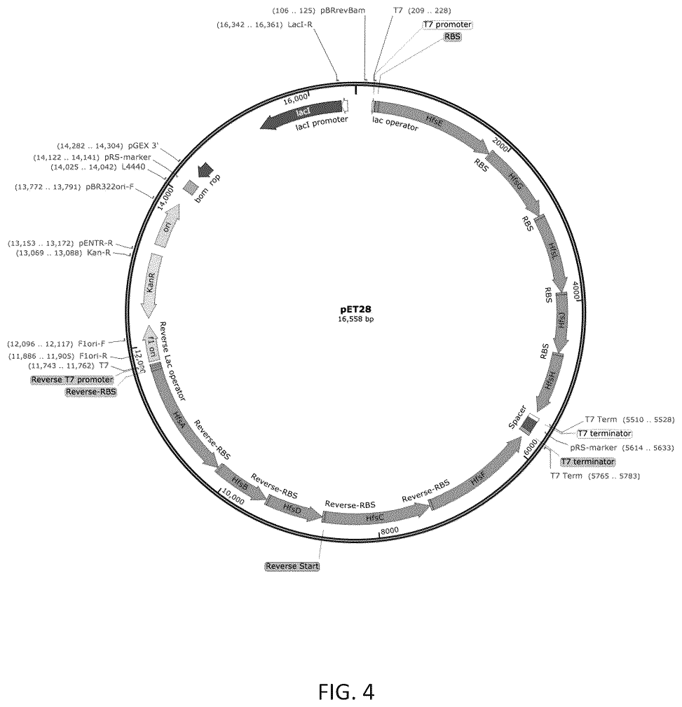

FIG. 4 shows an exemplary nucleic acid-plasmid construct. The engineered pET28 plasmid may be used for production of holdfast in E. coli, comprising genes for hfsA, hfsB, hfsC, hfsF, hfsE, hfsG, hfsL, hfsJ, and hfsH among others. The construct may comprise hfsE, hfsG, hfsL, hfsJ and hfsH in a forward or sense direction, and hfsA, hfsB, hfsD, hfsC, and hfsF in a reverse or antisense direction. The construct may comprise spacer regions, lac operator, lacI promoter, kanamycin R region, RBS, and other variable regions. The location of these regions may vary.

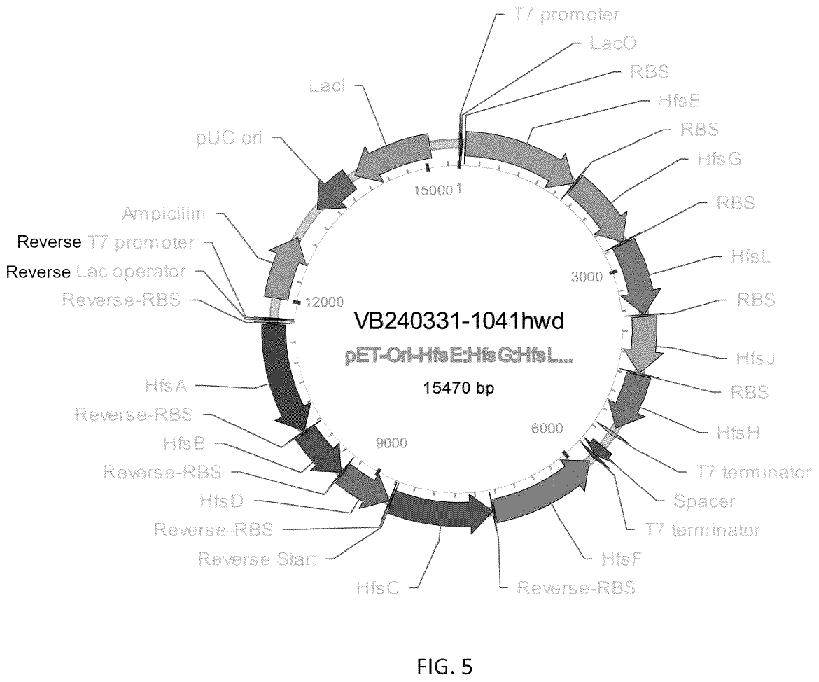

FIG. 5 shows an exemplary nucleic acid-plasmid construct. The engineered pET-Orl-Hfs plasmid may be used for production of holdfast in E. coli, comprising genes for hfsA, hfsB, hfsC, hfsD, hfsE, hfsF, hfsG, hfsL, hfsJ, and hfsH. The construct may comprise hfsE, hfsG, hfsL, hfsJ and hfsH in a forward or sense direction, and hfsA, hfsB, hfsD, hfsC, and hfsF in a reverse or antisense direction. The construct may comprise spacer regions, lac operator, lacI promoter, ampicillin, RBS, and other variable regions. The location of these regions may vary.

### Methods

### Methods of Producing Engineered Cells

Disclosed herein, in some embodiments, are methods of producing engineered cells. In some embodiments, the methods may comprise constructing a chimeric nucleic acid comprising (i) one or more nucleic acid sequences having at least 90% sequence identity to any one of SEQ ID NOs: 1-10 and (ii) a plurality of spacer sequences that separate the one or more nucleic acid sequences, wherein the plurality of spacer sequences having at least 90% sequence identity to any one of SEQ ID NOs: 33-35.

In some cases, the engineered cell may include one or more exogenous nucleic acid sequences that regulates production of a bioadhesive (e.g., holdfast). As an example, the one or more exogenous nucleic acid sequences may be any one of the sequences disclosed in Table 1. In some embodiments, the nucleic acid sequence includes one of the genes disclosed in Table 1. In some embodiments, the nucleic acid sequence includes two of the genes disclosed in Table 1. In some embodiments, the nucleic acid sequence includes three of the genes disclosed in Table 1. In some embodiments, the nucleic acid sequence includes four of the genes disclosed in Table 1. In some embodiments, the nucleic acid sequence includes five of the genes disclosed in Table 1. In some embodiments, the nucleic acid sequence includes six of the genes disclosed in Table 1. In some embodiments, the nucleic acid sequence includes seven of the genes disclosed in Table 1. In some embodiments, the nucleic acid sequence includes eight of the genes disclosed in Table 1. In some embodiments, the nucleic acid sequence includes nine of the genes disclosed in Table 1. In some embodiments, the nucleic acid sequence includes all ten of the genes disclosed in Table 1.

In some embodiments, the chimeric nucleic acid may include a spacer. In some embodiments, the method may comprise incorporating a spacer into the nucleic acid sequence between each of the genes incorporated into the chimeric nucleic acid. In some embodiments, the genes may be any one of the genes disclosed in Table 1. In some embodiments, the spacer may comprise a sequence as disclosed in Table 4. In some embodiments, the spacer is used to split the one or more nucleic acid sequences into two or more groups. In some embodiments, the spacer is used to split the 10 genes into two groups that are expressed in opposite directions. In some embodiments, the spacer is used between two groups of five holdfast genes to facilitate expression of a single holdfast gene. In some embodiments, the spacer enables the 10 genes to be expressed individually to make 10 holdfast proteins.

In some embodiments, the method may comprise inserting the nucleic acid in a plasmid of a base cell to construct an engineered plasmid. In some embodiments, the plasmid may comprise a pET28a(+) plasmid. In some embodiments, inserting the plasmid may comprise ligating the nucleic acid in the plasmid.

In some embodiments, the plasmid may be included in a base cell that does not produce a holdfast component. In some embodiments, the base cell may be transformed into a competent cell. In some embodiments, the competent cell comprises an E. coli cell. In some embodiments, the E. coli cell comprises an E. coli BL21 (DE3) competent cell. In some embodiments, the E. coli cell comprises an E. coli MG1655 cell. In some embodiments, the E. coli cell comprises an E. coli HMS174 cell. In some embodiments, the E. coli cell comprises an E. coli cell or strain that is engineered in-house.

### Methods of Producing Bioadhesives

Disclosed herein, in some embodiments, are methods for producing a bioadhesive. In some cases, the bioadhesive comprises holdfast. In some embodiments, the holdfast may be a glue produced by the methods described below. In some embodiments, the method may comprise providing an engineered cell as discussed herein, e.g., an engineered cell having one or more exogenous nucleic acid sequences that regulates production of a holdfast component, wherein the one or more exogenous nucleic acid sequences having at least 90% sequence identity to any one of SEQ ID NOs: 1-10.

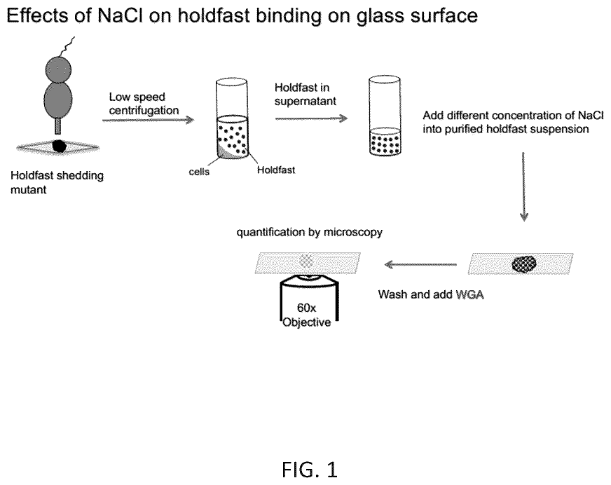

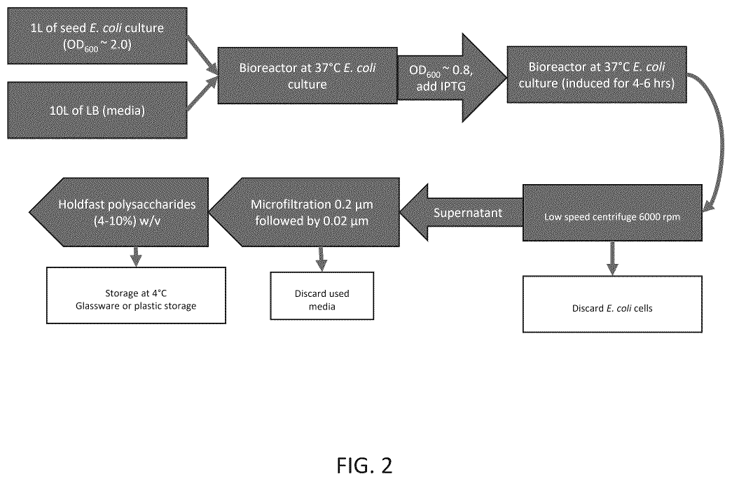

In some embodiments, the method may comprise subjecting the engineered cell to a medium that induces expression of the one or more exogenous nucleic acid sequences to produce a bioadhesive component. In some embodiments, the bioadhesive comprises a holdfast (e.g., produced using the methods described herein, such as those described with respect to FIGS. 1-2) In some embodiments, cells of E. coli strains (e.g., E. coli BL21) may be engineered to contain a holdfast plasmid. In some embodiments, the E. coli BL21 strains with holdfast plasmid can be grown in lysogeny broth (LB) at 37° C. In some embodiments, the medium comprises Isopropyl β-D-1-thiogalactopyranoside (IPTG). The IPTG may be added to the medium for 1 hour during the exponential growth phase of E. coli growth. The IPTG may be added to the medium for at least about 10, 20, 30, 40, 50, 60 or more minutes during the exponential growth phase of E. coli growth. The IPTG may be added to the medium for at most about 10, 20, 30, 40, 50, 60 or more minutes during the exponential growth phase of E. coli growth. The IPTG may be added to the medium for at least about 1 or 2 or more hours during the exponential growth phase of E. coli growth. The IPTG may be added to the medium for at most about 1 or 2 or more hours during the exponential growth phase of E. coli growth.

FIG. 1 depicts a schematic of holdfast binding to glass surfaces, the process of isolation of holdfast from cell culture, purification with different concentrations of salt, and quantification by microscopy. Holdfast is produced using mutant bacteria in a host cell. Centrifugation of the cells at low speeds separates the holdfast from cell debris in the medium. The supernatant is extracted, and holdfast is purified, using different concentrations of NaCl, into a suspension. After washing and adding wheat germ agglutinin (WGA), the holdfast molecules are quantified through microscopy.

FIG. 2 depicts an exemplary workflow for the method of producing holdfast polysaccharides using E. coli cultures. The steps involved include growth, induction, purification, filtration and harvesting of polysaccharides.

### One-Part Method:

In some embodiments, the method may comprise growing the E. coli strains mentioned herein in one or more cultures. In some embodiments, the E. coli strains may be grown in one or more overnight cultures. In some embodiments, the optical density of the one or more cultures are measured. In some embodiments, the one or more cultures are then diluted. In some embodiments, the one or more cultures are then diluted to an OD₆₀₀ of 0.4. In some embodiments, the diluted culture is grown further at 37° C. In some embodiments, the diluted culture is grown at 37° C. for 4 hours, to a maximum density of OD₆₀₀ of 2 to OD₆₀₀ of 0.6. In some embodiments, the diluted culture is grown at 37° C. for from 4 hours to 6 hours, to a maximum density of OD₆₀₀ of 2 to OD₆₀₀ of 0.6. In some embodiments, an inducer is added to the growing culture. In some embodiments, the inducer comprises Isopropyl β-D-1-thiogalactopyranoside (IPTG). In some embodiments, the induction lasts for at least 1 hour at 37 C. In some embodiments, the induction lasts for at least 4 hours at 37 C. In some embodiments, the inducer is added to the medium for at least about 10, 20, 30, 40, 50, 60 or more minutes during the exponential phase of E. coli growth. In some embodiments, the inducer is added to the medium for at most about 10, 20, 30, 40, 50, 60 or more minutes during the exponential phase of E. coli growth. In some embodiments, the inducer is added to the medium for at least about 2 hours during the exponential growth phase of E. coli growth. In some embodiments, the inducer is added to the medium for at least about 3 hours during the exponential growth phase of E. coli growth. In some embodiments, the inducer is added to the medium for at least about 4 hours during the exponential growth phase of E. coli growth. In some embodiments, the inducer is added to the medium for over 4 hours during the exponential growth phase of E. coli growth. In some embodiments, the induction comprises incubation at 37° C., shaking in a shaker, or both. In some embodiments, this method leads to glue production in the bacteria. In some embodiments, this glue is secreted into the media.

In some embodiments, the method may comprise purifying the holdfast from the medium. In some embodiments, purifying comprises centrifuging and isolating the holdfast from the medium. In some embodiments, this purification results in production of a glue suspension. In some embodiments, this purification comprises using centrifuge speeds of 4000-6000 pm. In some embodiments, the cells are discarded and the glue suspension is concentrated to a desired concentration. In some embodiments, the glue suspension is concentrated using microfilters. In some embodiments, the microfilters include 0.22μ microfilters. In some embodiments, the final concentration of the glue in the glue suspension is about 4% to about 10%. In some embodiments, the final concentration of the glue in the glue suspension is at least about 4%. In some embodiments, the final concentration of the glue in the glue suspension is at most about 10%. In some embodiments, the final concentration of the glue is about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the final concentration of the glue is about 4%, about 5%, about 6%, about 7%, about 8%, about 9%, or about 10%.

In some embodiments, the glue suspension is packaged in lysine coated containers.

### Two-Part Method:

In some embodiments, the method comprises two parts: harvesting polysaccharides and producing deacetylase (with HfsH as a crosslinker). In some embodiments, the method comprises growing one or more cultures of the E. coli strains mentioned herein. In some embodiments, the one or more cultures are overnight cultures. In some embodiments, the E. coli contain all genes except deacetylase required to produce the non-adhesive glue. In some embodiments, the optical density of the culture is measured. In some embodiments, the overnight culture is then diluted. In some embodiments, the overnight culture is then diluted to an OD₆₀₀ of 0.4. In some embodiments, the diluted culture is grown further at 37° C. In some embodiments, the diluted culture is grown at 37° C. for 4 hours, to a maximum density of OD₆₀₀ of 2 to OD₆₀₀ of 0.6.

In some embodiments, an inducer is added to the growing culture. In some embodiments, the inducer comprises Isopropyl β-D-1-thiogalactopyranoside (IPTG). In some embodiments, the induction lasts for at least 1 hour at 37° C. In some embodiments, the induction lasts for at least 4 hours at 37° C. In some embodiments, the inducer is added to the medium for at least about 10, 20, 30, 40, 50, 60 or more minutes during the exponential phase of E. coli growth. In some embodiments, the inducer is added to the medium for at most about 10, 20, 30, 40, 50, 60 or more minutes during the exponential phase of E. coli growth. In some embodiments, the inducer is added to the medium for at least about 2 hours during the exponential growth phase of E. coli growth. In some embodiments, the inducer is added to the medium for at least about 3 hours during the exponential growth phase of E. coli growth. In some embodiments, the inducer is added to the medium for at least about 4 hours during the exponential growth phase of E. coli growth. In some embodiments, the inducer is added to the medium for over 4 hours during the exponential growth phase of E. coli growth. In some embodiments, the induction comprises incubation at 37° C., shaking in a shaker, or both. In some embodiments, the method comprises adding new media to the culture after induction, to a final OD₆₀₀ of 0.6. In some embodiments, this method leads to non-adhesive glue (e.g., in the form of non-adhesive glue polysaccharides) production by the bacteria. In some embodiments, this non-adhesive glue is secreted into the media. In some embodiments, the glue suspension remains non-adhesive until addition of deacetylase enzyme.

In some embodiments, the method may comprise purifying the non-adhesive glue from the medium to form a glue suspension. In some embodiments, this purification is done by removing the bacteria using an ultracentrifuge. In some embodiments, this purification results in production of a glue suspension. In some embodiments, this purification comprises using centrifuge speeds of 4000-6000 pm. In some embodiments, the cells are discarded and the glue suspension is concentrated to a desired concentration. In some embodiments, the glue suspension is concentrated using microfilters. In some embodiments, the microfilters include 0.22μ microfilters. In some embodiments, the final concentration of the glue in the glue suspension is about 4% to about 10% by weight. In some embodiments, the final concentration of the glue in the glue suspension is at least about 4%. In some embodiments, the final concentration of the glue in the glue suspension is at most about 10%. In some embodiments, the final concentration of the glue in the glue suspension is about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the final concentration of the glue in the glue suspension is about 4%, about 5%, about 6%, about 7%, about 8%, about 9%, or about 10%.

In some embodiments, the non-adhesive glue is packaged in regular (e.g., plastic, cardboard, or metal) containers.

In some embodiments, the non-adhesive glue and HfsH enzymes are combined before application to activate the adhesiveness of the glue. In some embodiments, the non-adhesive glue and HfsH enzymes are combined five minutes before application to form an adhesive glue suspension. In some embodiments, the adhesive glue suspension can be used in one or more 3D printing processes described below.

In some embodiments, HfsH enzymes are produced using the methods described herein. In some embodiments, the E. coli cells are lysed and affinity chromatography is used to purify the enzyme.

In some embodiments, HPLC is used to detect amount of each monomer that has been produced. In some embodiments, a fluorescent lectin dye is used to quantify how much glue has been produced.

In some embodiments, the holdfast may comprise a force of adhesion of about 70 Newtons per square millimeter (N/mm²). In some embodiments, the holdfast may comprise a force of adhesion of at least about 20, 25, 30, 35, 40, 45, 50, 55, 60, 65, 70, 75, 80, 85, 90, 95, 100, 105, 110, 115, 120, 125, 130, 135, 140, 145, or 150 N/mm². In some embodiments, the holdfast may comprise a force of adhesion of at most about 20, 25, 30, 35, 40, 45, 50, 55, 60, 65, 70, 75, 80, 85, 90, 95, 100, 105, 110, 115, 120, 125, 130, 135, 140, 145, or 150 N/mm². In some embodiments, the holdfast may comprise a force of adhesion of 20 to about 150 N/mm². In some embodiments, the holdfast may comprise a force of adhesion of 25 to about 145 N/mm². In some embodiments, the holdfast may comprise a force of adhesion of 30 to about 140 N/mm². In some embodiments, the holdfast may comprise a force of adhesion of 35 to about 135 N/mm². In some embodiments, the holdfast may comprise a force of adhesion of 40 to about 130 N/mm². In some embodiments, the holdfast may comprise a force of adhesion of 45 to about 125 N/mm². In some embodiments, the holdfast may comprise a force of adhesion of 50 to about 120 N/mm². In some embodiments, the holdfast may comprise a force of adhesion of 55 to about 115 N/mm². In some embodiments, the holdfast may comprise a force of adhesion of 60 to about 110 N/mm². In some embodiments, the holdfast may comprise a force of adhesion of 65 to about 105 N/mm². In some embodiments, the holdfast may comprise a force of adhesion of 60 to about 80 N/mm². In some embodiments, the holdfast may comprise a force of adhesion of 65 to about 75 N/mm².

### Applications

Disclosed herein, in some embodiments, are uses for the bioadhesives (e.g., the holdfast produced according to the methods described herein) disclosed herein. In some embodiments, the bioadhesives disclosed herein may be used in medical applications, including but not limited to uses as: tissue adhesives, hemostatas, tissue sealants, functional wound dressings, medical device fixation, medical sutures (e.g., a replacement for medical sutures), and a replacement for traditional drug dosage systems. In some embodiments, the bioadhesives disclosed herein may be used in healthcare applications, including but not limited to bone and tissue grafting, dental repair and fillers, wound dressing tape, band-aids, internal and external suture replacements, dissolvable and removable sutures, permanent sutures, and pacemakers.

In some embodiments, the bioadhesives disclosed herein may be used in industrial applications, including but not limited to drywall, plywood, OSB, plumbing, adhesive tape, consumer-level glue, material binders, shoe and fabric binders, 3D organic material binders, vessel sealants, insulation tape, foamed tape, graphene binding, batteries (cathode and anode), primer replacement, multi-step glue applications such as in shoes (adhering the lower part of the shoe to the upper), polyolefins, TPUs, PTFEs, Teflon, and others.

In some embodiments, the bioadhesives disclosed herein may be used in three-dimensional (3D) printing. In some cases, the 3D printing may comprise powder-based printing, fused deposition modeling (FDM), and stereolithography (SLA).

3D printing systems may include a substrate on which a 3D object is to be printed. The process may include providing a layer of material for use in making the 3D object. The process may then include providing a binder layer that will be used to bind the initial layer of material to subsequent layers of material. The process may be repeated until the 3D object is finished printing. The bioadhesive disclosed herein may be used as a binder in the binder layer to bind layers of 3D printed materials together to form 3D printed objects (e.g., for use in one or more applications as described above.)

In some cases, the bioadhesive provided herein may be used as a binder in 3D printing processes. In some embodiments, the 3D printing processes may comprise mixing the bioadhesive with printing materials. In some cases, the printing materials may comprise powder materials. In some cases, the printing materials may comprise metal. In some cases, the printing materials may comprise a polymeric precursor. In some cases, the bioadhesive may be present in the mixture for at about at least 1 wt %, at least about 2 wt %, at least about 5 wt %, at least about 10 wt %, at least about 20 wt %, at least about 30 wt %, or at least about 40 wt %. In some cases, the bioadhesive component may be present in the mixture for at most about 40 wt %, at most about 30 wt %, at most about 20 wt %, at most about 10 wt %, at most about 5 wt %, at most about 2 wt %, or at most about 1 wt %.

In some cases, the 3D printing may produce a 3D structure in a layer-by-layer fashion by using light or heat to selectively cure precursors into a cured material or object.

In some cases, the mixture may further comprise an initiator. In some cases, the initiator may comprise a thermal initiator. In some cases, the initiator may comprise a photoinitiator.

The bioadhesive component may act as a binder to create a free standing uncured object. The uncured object may be cured by light or heat to create a hard cured object. In some cases, the bioadhesive may be removed after the 3D printing, e.g., by heating or solvent elution.

The mixture may comprise a photoactive resin to form a polymeric material. The photoactive resin may comprise a polymeric precursor of the polymeric material. The photoactive resin may comprise one or more photoinitiators configured to initiate formation of the polymeric material from the polymeric precursor. The viscosity of the photoactive resin may range from about 1 cP to about 2,000,000 cP.

The polymeric precursor in the photoactive resin may comprise monomers to be polymerized into the polymeric material, oligomers to be cross-linked into the polymeric material, or both. The monomers may comprise one or more of hydroxyethyl methacrylate; n-Lauryl acrylate; tetrahydrofurfuryl methacrylate; 2, 2, 2-trifluoroethyl methacrylate; isobornyl methacrylate; polypropylene glycol monomethacrylates, aliphatic urethane acrylate (i.e., Rahn Genomer 1122); hydroxyethyl acrylate; n-Lauryl methacrylate; tetrahydrofurfuryl acrylate; 2, 2, 2-trifluoroethyl acrylate; isobornyl acrylate; polypropylene glycol monoacrylates; trimethylpropane triacrylate; trimethylpropane trimethacrylate; pentaerythritol tetraacrylate; pentaerythritol tetraacrylate; triethyleneglycol diacrylate; triethylene glycol dimethacrylate; tetrathyleneglycol diacrylate; tetrathylene glycol dimethacrylate; neopentyldimethacrylate; neopentylacrylate; hexane dioldimethacylate; hexane diol diacrylate; polyethylene glycol 400 dimethacrylate; polyethylene glycol 400 diacrylate; diethylglycol diacrylate; diethylene glycol dimethacrylate; ethyleneglycol diacrylate; ethylene glycol dimethacrylate; ethoxylated bis phenol A dimethacrylate; ethoxylated bis phenol A diacrylate; bisphenol A glycidyl methacrylate; bisphenol A glycidyl acrylate; ditrimethylolpropane tetraacrylate; and ditrimethylolpropane tetraacrylate.

The photoinitiator may be present from about 0.1 wt % to about 10 wt % in the mixture. The photoinitiator may comprise one or more of benzophenones, thioxanthones, anthraquinones, benzoylformate esters, hydroxyacetophenones, alkylaminoacetophenones, benzil ketals, dialkoxyacetophenones, benzoin ethers, phosphine oxides acyloximino esters, alphahaloacetophenones, trichloromethyl-S-triazines, titanocenes, dibenzylidene ketones, ketocoumarins, dye sensitized photoinitiation systems, maleimides, and mixtures thereof.

In some cases, the 3D printing may use metal powders. The metal powders may comprise one or more of aluminum, calcium, magnesium, barium, scandium, titanium, vanadium, chromium, manganese, iron, cobalt, nickel, copper, zinc, yttrium, niobium, molybdenum, ruthenium, rhodium, silver, cadmium, actinium, and gold.

In some cases, the 3D printing may use ceramic materials. The ceramic materials may comprise metal (e.g., aluminum, titanium), non-metal (e.g., oxygen or nitrogen), and/or metalloid (e.g., germanium, silicon) atoms primarily held in ionic and covalent bonds. The ceramic materials may comprise one or more of aluminide, boride, beryllia, carbide, chromium oxide, hydroxide, sulfide, nitride, mullite, kyanite, ferrite, titania zirconia, yttria, and magnesia.

The bioadhesive may act as a binder to create a free-standing unfused object. The unfused object may be subject to high temperature sintering to fuse the metal powders or ceramic materials to form a hard object. The bioadhesive component may be removed during the sintering process.

In some cases, the 3D printing processes may comprise mixing the bioadhesive with one or more food sources, one or more enzyme-producing bacteria, one or more nitrogen sources, and one or more calcium sources. The one or more food sources may comprise the qualities of a food source for the enzyme-producing bacteria. The 3D printing processes may further comprise loading a 3D file that defines an object to be printed onto a 3D printer processes and printing the object to be printed using the 3D printing composition. The 3D printing processes may further comprise keeping the resulting 3D printed object moist until the natural cement hardening time has elapsed. The 3D printing processes provided herein can objects for use in the applications noted above, such as sinks, faucets, light fixtures, furniture, and aircraft interiors.

In some embodiments, the glue binds with metal powder, cures almost as hard as aluminum, and easily vaporizes when put into a sintering furnace at a much lower temperature than what is currently available.

In some embodiments, the two-part method described herein is similar to an epoxy and allows for easier applications. In some embodiments, the glue could be pushed through a tube for 3D printing and then activated at the source, rather than in the tubing. In some embodiments, for healthcare suture applications, the glue could be non-sticky and applied to the cut, allowing the surgeon time to decide when to activate the glue to close the wound. In some embodiments, for bone grafting, the glue could allow for proper alignment prior to curing.

### EXEMPLARY EMBODIMENTS

Among the exemplary embodiments are.
1. An engineered cell comprising one or more exogenous nucleic acid sequences that regulates production of a holdfast component, wherein the one or more exogenous nucleic acid sequences comprises at least 90% sequence identity to any one of SEQ ID NOs: 1-11.
2. The engineered cell of embodiment 1, wherein the one or more exogenous nucleic acid sequences encodes one or more proteins that regulate production of the holdfast component.
3. The engineered cell of embodiment 1, wherein the holdfast component comprises a polysaccharide.
4. The engineered cell of embodiment 1, wherein the one or more exogenous nucleic acid sequences are derived from Caulobacterales bacterium.
5. The engineered cell of embodiment 4, wherein the Caulobacterales bacterium comprises Hirschia baltica bacterium or Caulobacter crescentus bacterium.
6. The engineered cell of embodiment 1, wherein the one or more exogenous nucleic acid sequences are contained in a plasmid of the engineered cell.
7. The engineered cell of embodiment 1, comprising ten exogenous nucleic acid sequences with at least 90% sequence identity to SEQ ID NOs: 1-10 respectively.
8. The engineered cell of embodiment 1, further comprising a spacer sequence with at least 90% sequence identity to SEQ ID NO: 12 between one or more groups of the one or more exogenous nucleic acid sequences.
9. The engineered cell of embodiment 1, wherein the one or more exogenous nucleic acid sequences comprises an exogenous nucleic acid sequence comprising at least 90% sequence identify to SEQ ID NO: 11.
10. The engineered cell of embodiment 1, wherein the engineered cell is engineered from an E. coli competent cell.
11. The engineered cell of embodiment 10, wherein the engineered cell comprises an E. coli BL21 (DE3) competent cell.
12. The engineered cell of embodiment 1, wherein the one or more exogenous nucleic acid sequences is attached to one or more ribosome binding sites (RBSs).
13. A method of producing an engineered cell, comprising
   a. constructing a nucleic acid comprising (i) one or more nucleic acid sequences comprising at least 90% sequence identity to any one of SEQ ID NOs: 1-11;
   b. inserting the nucleic acid in a plasmid of a base cell to construct an engineered plasmid; and
   c. transforming the engineered plasmid to an E. coli competent cell to generate the engineered cell.
14. The method of embodiment 13, wherein the one or more exogenous nucleic acid sequences comprises ten exogenous nucleic acid sequences with at least 90% sequence identity to SEQ ID NOs: 1-10 respectively.
15. The method of embodiment 13, wherein the nucleic acid further comprises a spacer sequence with at least 90% sequence identity to SEQ ID NO: 12 between one or more groups of the one or more exogenous nucleic acid sequences.
16. The method of embodiment 13, wherein the one or more exogenous nucleic acid sequences comprises an exogenous nucleic acid sequence comprising at least 90% sequence identify to SEQ ID NO: 11
17. The method of embodiment 13, wherein the E. coli competent cell comprises an E. coli BL21 (DE3) competent cell.
18. The method of embodiment 13, wherein b) comprises ligating the nucleic acid in the plasmid.
19. The method of embodiment 13, wherein the one or more nucleic acid sequences regulates production of a holdfast component.
20. The method of embodiment 19, wherein the base cell does not produce the holdfast component.
21. A method of producing a holdfast component, comprising
   a. providing an engineered cell of any one of embodiments 1-20; and
   b. subjecting the engineered cell to a medium that induces expression of the one or more exogenous nucleic acid sequences to produce the holdfast.
22. The method of embodiment 21, wherein the medium comprises Isopropyl R-D-1-thiogalactopyranoside (IPTG).
23. The method of embodiment 21, further comprising purifying the holdfast from the medium.
24. The method of embodiment 23, wherein purifying comprises centrifuging and isolating the holdfast from the medium.
25. Use of the holdfast produced in any one of embodiments 21-24 as an adhesive in one or more industrial or medical applications.
26. The use of embodiment 25, wherein the industrial application comprises three dimensional (3D) printing.
27. The use of embodiment 26, wherein the holdfast is used as a binder in 3D printing.
28. A bacteria cell that has been modified to secrete a holdfast faster and/or in higher quantity than a wildtype bacteria cell.
29. The bacteria cell of embodiment 28, wherein the wildtype bacteria cell comprises an E. coli cell.

### Examples

The following examples are included for illustrative purposes only and are not intended to limit the scope of the invention.

### Example 1: Holdfast Production in an Escherichia coli (E. coli) System

A plasmid was engineered with holdfast genes from Hirschia baltica and was optimized to synthesize holdfast in an E. coli system. Ten holdfast proteins were identified that are required to synthesize holdfast from the bacterium Hirschia baltica. New artificial ribosome binding sites (RBS) were added to each gene sequence (only the coding region from the bacterium) and the artificial DNA sequence was generated using de novo DNA synthesis technologies (GeneScript). In addition, spacer sequences were added to separate the individual genes (see Table 3 disclosing SEQ ID NO. 12). The synthesized DNA sequence with holdfast genes were inserted into pET28a(+) plasmid and ligated to make a ligated plasmid including the 10 gene sequences with the spacer in between in gene. This plasmid was then transformed into E. coli BL21 (DE3) competent cells. Holdfast expression was induced by the addition of IPTG, which was added to the media for 1 hour during the exponential phase of E. coli growth. Holdfast was purified by centrifuging out the cells.

### Example 2: Holdfast Production in an E. coli System Using Modified HsfH Gene from Hirschia baltica

A polysaccharide deacetylase enzyme, HfsH, that modulates holdfast binding properties was engineered. Results from the engineered HfsH indicate that the level of activity of HfsH correlates with adhesiveness of holdfast polysaccharides. Mutations were introduced in the different part of the enzyme in order to create better, more hyperactive, and more stable enzymes. The natural sequence was modified to artificial sequences for E. coli and Yeast expression systems. A plasmid was engineered with mutated hfsH gene from Hirschia baltica and optimized to modify holdfast polysaccharides in an E. coli system. Important residues were identified that are required holdfast production activity (deacetylation of GlcNAc polysaccharides). New artificial ribosome binding sites (RBS) were added to each gene sequence (only the coding region from the bacterium) and the artificial DNA sequence was generated using de novo DNA synthesis technologies (Allozyme). The synthesized DNA sequence with the engineered hfsH gene were inserted into pET28a(+) plasmid and ligated to make pET28-hfsH plasmid. This plasmid was then transformed into E. coli BL21 (DE3) competent cells. Holdfast expression was induced by the addition of IPTG, which was added to the media for 1 hr during the exponential phase of E. coli growth. HfsH was purified by lysing the cells, centrifuging out the debris and using chromatography to purify HfsH.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

### Example 3: Holdfast Production in an E. coli System Using Modified HsfH Gene From Caulobacter crescentus

A polysaccharide deacetylase enzyme, HfsH, that modulates holdfast binding properties was engineered. Results from the engineered HfsH indicate that the level of activity of HfsH correlates with adhesiveness of holdfast polysaccharides. Mutations were introduced in the different part of the enzyme in order to create better, more hyperactive, and more stable enzymes. The natural sequence was modified to artificial sequences for E. coli and Yeast expression systems. A plasmid was engineered with mutated hfsH gene from Caulobacter crescentus and optimized to modify holdfast polysaccharides in an E. coli system. Important residues were identified that are required holdfast production activity (deacetylation of GlcNAc polysaccharides). New artificial ribosome binding sites (RBS) were added to each gene sequence (only the coding region from the bacterium) and the artificial DNA sequence was generated using de novo DNA synthesis technologies (Allozyme). The synthesized DNA sequence with the engineered hfsH gene were inserted into pET28a(+) plasmid and ligated to make pET28-hfsH plasmid. This plasmid was then transformed into E. coli BL21 (DE3) competent cells. Holdfast expression was induced by the addition of IPTG, which was added to the media for 1 hr during the exponential phase of E. coli growth. HfsH was purified by lysing the cells, centrifuging out the debris and using chromatography to purify HfsH.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. An engineered cell comprising one or more exogenous nucleic acid sequences that regulates production of a bioadhesive, wherein the one or more exogenous nucleic acid sequences comprise a sequence at least 90% sequence identical to any one of SEQ ID NOs: 1-10 and 42-51.

2. The engineered cell of claim 1, wherein the one or more exogenous nucleic acid sequences encodes one or more proteins that regulate production of the bioadhesive.

3. The engineered cell of claim 1, wherein the bioadhesive comprises a polysaccharide.

4. The engineered cell of claim 1, wherein the one or more exogenous nucleic acid sequences are derived from Caulobacterales bacterium.

5. The engineered cell of claim 4, wherein the Caulobacterales bacterium comprises Hirschia baltica bacterium or Caulobacter crescentus bacterium.

6. The engineered cell of claim 1, wherein the one or more exogenous nucleic acid sequences are contained in a plasmid of the engineered cell.

7. The engineered cell of claim 1, wherein the one or more exogenous nucleic acid sequences comprises ten exogenous nucleic acid sequences, wherein each of the ten exogenous nucleic acid sequences has at least 90% sequence identity to one of SEQ ID NOs: 1-10 and 42-51.

8. The engineered cell of claim 1, further comprising one or more spacers, wherein the one or more spacers comprises a sequence with at least 90% sequence identity to any one of SEQ ID NOs: 33-35, wherein the one or more spacers are between one or more groups of the one or more exogenous nucleic acid sequences.

9. The engineered cell of claim 1, further comprising one or more promoters, wherein the one or more promoters comprise a sequence that has at least 90% sequence identical to any of SEQ ID NOs: 11-30 and 38.

10. The engineered cell of claim 1, wherein the engineered cell is engineered from an E. coli competent cell.

11. The engineered cell of claim 10, wherein the engineered cell comprises an E. coli BL21 (DE3) competent cell.

12. The engineered cell of claim 1, further comprising one or more ribosome binding sites (RBSs) attached to the one or more exogenous nucleic acid sequences.

13. The engineered cell of claim 1, wherein the bioadhesive comprises a holdfast component.

14. (canceled)

15. (canceled)

16. (canceled)

17. (canceled)

18. (canceled)

19. (canceled)

20. (canceled)

21. (canceled)

22. (canceled)

23. A method of producing a bioadhesive, comprising
a. providing an engineered cell of claim 1; and
b. subjecting the engineered cell to a medium that induces expression of the one or more exogenous nucleic acid sequences to produce the bioadhesive component.

24. The method of claim 23, wherein the medium comprises Isopropyl β-D-1-thiogalactopyranoside (IPTG).

25. The method of claim 23, further comprising purifying the bioadhesive from the medium.

26. The method of claim 25, wherein purifying comprises centrifuging and isolating the bioadhesive from the medium.

27. Use of the bioadhesive produced in claim 23 as an adhesive in one or more applications.

28. The use of claim 27, wherein the application comprises three-dimensional (3D) printing.

29. The use of claim 28, wherein the bioadhesive is used as a binder in the 3D printing.

30. (canceled)

31. (canceled)


SEQUENCE LISTING (51 sequences)

SEQ ID NO: 1 - DNA, 1554 bp, Hirschia baltica
  source 1..1554: mol_type = genomic DNA
atgtttaaatttgtaatggaaatcgctggcatgagcctcgacgctatggatccggacaataatgcaagtaacgatacatatcgtattgcaaaattgcgccagcacaaaagcgaaaaacacaaattacacaatacacaacctgatcaacccgccaaacgttttccaatagatcgctctggtctacgttttatactgcgcatatttgattttaccgccggctttgcaattattcttattgcttgtcatcttttaaaaattgatctgctaagcgaaaaccttagaacttctctgccctacatagcagcgccattatttgccttcttgggcatgcgtataacgggtgcctatcgttttgcatttgatgaacatccacttacacacattggacgtgttttgcttggctcaagccttggggtggcacttgttcacatcattagtttggttttcaatcttggtgatgatcagctttaccaaatttctgcaattgttttgtgtggctatttgggattgcacgcgcattacgccgctcttcttcgttccctcacgcgttctggcgcgcttgcagataatgtcgttatcgtcggtgcgacccctgctgcttttgatttaatcgaaaaaaatcgcaaaaaacgtgagatgaatattctaggtgtctttgaagacaggctagatcgcgcacccgcagcaatcgcggatgttccggtcattgggaaagtcgatgatctactcgaatgggataaacttcctgatgtcgacagaattatcctaacagtgacatcaaccgcacaagaacgtgttcgtacccttatagatcgtctacgccttttgcctcaagaagtgattctcatgctggacctagacgggtttagtccagagaaaacctcaatcgccaatatggtcgatacacccgcagcttatgtttcaggtgcgcccaaagatgtcagacgtgccgcgatcaaacgcggtttagatattgtggttgcaagcgccatgctcatacttttcttgccatttatgatgatcattgcaactttgatcaaactagatagccaaggctcaattttctttagacagcgacgtcatggttttaacaatcagattattcgtgtctggaaatttcggactatgcacccggataaagcggctgaagatggcacaaaagtcatccaaaccgtgagcaatgataaacgtgtcacgcgcattggtgccttcttacgtcgcacaagtttggatgaacttccccaactcataaatattctagttggagacatgtcacttgtcggccctcgcccccatgccgttggcatgacaaccgaggaagttgaagtccacaatatcgtcgctgaatacgctcatcgtcacagaatgaaaccaggcttaaccggttgggcacaaatcaatggatctcgcggcccagtccacacagcagagcttgttaaagatcgcgtgcgtttggacatggaatatatggaaaaagcatcattctggtttgatctatacgtcatcctgatgactgctccctgccttttgggcgactcaaaaacacagcgctag

SEQ ID NO: 2 - DNA, 992 bp, unidentified
  source 1..992: mol_type = genomic DNA
atgaacacaacgccccaacttagcgccaacccacccgttcgcgacacgacaaccccacgcgaaatcgtggtcgataacccgcactggaaagacaaggcaaaccaaaagctgtctattcttgtacctagctataaagacgatccagaagctttattggcctcattatccaaatgcaaaaatgtcgaagacatcgaatttatttttatgatgatggcggcggagaccgcacacttattgagaaaatttcggcgcacgcagctgaggccagctttccagttcggatcatctctgcacaacacaatattggacgtgcaggcgcgcgaaatcgtttattgaactatgcgcgatgcaaatgggtattgctccttgatgccgacatgcttcccgataacgaagatttcctgactaattacatccaagaaatatcagttcatcccgaccctaaacttatcgtcggcgggttttcacttaagcaagcctcaacggccccagaatatgctttgcatcggtggcaagctgaaaaatcagaatgcatgtcttctaagctgcgcaacacagaacctgggcgttatgttttcacaagtaatgttcttgcccatgcagacctgtttgaagaagtcccttttgatgaaacattcagtggatggggctgggaagatgtcgattggggattgcgtatcgccaagacttatctagttctccacatagataatacagccactcatttaggccttgatactgatcgagatctcatgaaaaaatatggcaaatcgggtgctaattttaaaaaagcgattgaaaaacatcccgacgctttaaaatcaacaagtctatataaagttgctaacaagctagcgccgataccattcaagcccgtgatcaaatccataactggtagcattgcgacagcgcacttttttccaataaaggttagaggtctcgccctaaaattatggcgtgcaaccatatatgcggaggctcttcatgattga

SEQ ID NO: 3 - DNA, 986 bp, unidentified
  source 1..986: mol_type = genomic DNA
atgaaccaatccgtaaaagtcagtattgttattccaacatttcgccgcaagacggtttggaaaatgccattaccagcattctgaatatgacggatgctgctttggaaacggctgaaatcgttgtcgcagacaactcacccgaagccggcgccaaatccacagtagaacaactcaaactcaacactagcgtaccaataaactatgtcagtgaacccactccgggcgtatcaaatgcacgaaatgctggtctagctgtagcaaattctcgtctaattgccttcattgacgatgatgaaactgcccaaaatggatggctagataatttagtcgcagctcatcaaaagctcggtgccgctgtcatttttggccctgtcgaaactgtgcttccaaccgacacgcctcccgcgcacaaacaatatctggaagattttttctctcgcaaaggcccaagcgaaaccaaactaattgatgaagcttttggctgtggaaatgctttccttgatttggatcgcattcaaccttgcctccctgaaaatgagccgtttttcaataagatagccaatgaaactggcggcgaagatgactatctttttgctcgtgtgaaacagcatggtgaaacatttggctgggcacataatgcaattgttgatgaacacgtccccgaaaaacgtgcacatttaggttatacactgaggcgtgcattcgcctatggacaggggccctccaccaattgctggcgcaataatgacggcattgatattccacgcctattgatgtggatgattatcgggacaggtcagtttattatctatggaatctctgccttagcgatgtttgcaatcaagcatcccaaacgcgcatatatgttggataaagctgtaagaggactaggaaaacccctctggtttccaccatttattctgcaattttatggggatattcccgataagaaaaagcgcaaaaaagttatcaaaagcgcaacttaa

SEQ ID NO: 4 - DNA, 747 bp, unidentified
  source 1..747: mol_type = genomic DNA
atgtcgtccggtaagaaaatttacgacatgctagaacatttaacggttcctgaaacggaagcgaatgtggatgcattgctaaatggattgccggatgcgacttcacctcaggtgatttcgtttgtgaatgcacacgctgtaaatttgatggtgaaagatgagggcttgttcaaagcgctgatcggatcggatattttattgcgagatggttcaggcatgaagattttaatgaaatggctaaaccaaaatccgggtgcaaacctgaatggtacagatctaataccacgcattattgagaaatttgatggtatgaaggtcgctgtatttggaactcaggagccttggttgtctaaaggctgtgatgtcattgaaacgcgtggtggaacaatcgtatcgcgtttgaatggtttccaagatgaagcagcttatattgaagcgattgagacctctaaacccgaccttgtgattcttgctatgggaatgcccaagcaagaaatgacttctatggctttgcgggcagcagcgagctggccgacgacaatagtaaatggtggagctattattgatttccttgctgaacgcgttaaccgcgcaccagaaacttggcgaaagctgggtatggaatggctctaccgtctcattcaagaacctaaacgactattcggccgatacgtggttggaaatgtcatttttctcacgcgtggtttgatattatgtgtgacccaagctaatcccaaaataacttaa

SEQ ID NO: 5 - DNA, 768 bp, unidentified
  source 1..768: mol_type = genomic DNA
atgattgattggcattacacaccctctcgtacccttcccgccaaattaaaacgccgcatgacacaatggagacatgctgcgccagttgatgtgagcaacacacaatttcatgtatcatacacatttgatgattttccaatgtcggcggtcaatggtgccgacatcctcgaatctcatgatgggcacgcagcattttatgcctgcacaaaaatgataggcacacacggcgcatatggggacatgtacgatatcaaaaccatgttggaccttgaaaaccgtggacatgaaattggcgcgcatacccacagtcaccttgattgcgcgcaaagcaaacgtgaaactgtcttaaatgatattgacgcaaatatttctgcgctcatggaagccggcttaaaaaaacggcccacaagttttgcatacccttatggagaaaccctttttgataccaaaaaagaagtattcaaaaagtttgatctgtgtcgcggtattcttcccggaattaatgttggaaaagttgatctcgctcaattgcggtgttttgaactaaacgaaaatccagccacacgcattcgagcaataaatgctatcgaagaagcaggcaaaactggcggctgggtgattatatttacacatgatgtatcacctcagccaactgcgtatggaaccacaactgggatcgtcgaagaattgtgccaactgtctaaagcagcaggcgcaacgctttcaacgccaactgaggcagcaagaagctacggcctaatctcatga

SEQ ID NO: 6 - DNA, 1488 bp, unidentified
  source 1..1488: mol_type = genomic DNA
atgatgcaagacaatcaagccacccaaatgcctgtccggcgcgaacaacgcggtgtgaatttacgcgttcgtccacgttttggcgtgggcgatatattcttgcaattgtggcgatcactttggattatgatattggtgtttctgcctatcgtgatactgggtgttctgtttgctatgaccatgcccaaaacatatacggcattcggcacaatgcaggtcacgttggataagcaatatatttatgacccgcttgtcggagatgctggacgtggtgtttctattgaaactgaggcaattgttagtgcggaagctgagaaagccaattcccctatattagcgcgtcgcgttatggatcagatggggattggacacatctatcctaaaattgcccaagagatagcagaaacgaatgaccctgacaaaattcgaaaattggaagcctctgcatatgatgctttgaaccaaaattttggtgcatctcacggggtgaaatcacccttgcttacttttgtctttaagcatgaagatccaattgttgccgccgaggttgtcaataaatttctggaacaatttgtcgactatagagaaattcaaagtgatcaagatgatattgcggctgtggctggtcaaagatatcttgtgggtgaggccttaagtgaagccgaatcattgctacgcgacttccttgttgaaaatgaaattggtgaatttgatactgatcgattgtctgttggcacgacattggtgaatttacgcaatgaacttttgaccgttgaggcattggtaaaagaaggtgaaggtcgtttgagtggcttgcgtgctatgctgccaactacgccagaaacgattgcgctagaagttgagacaaatgcatcacaacgtgtgctggacctccagttagagcgtgagcaattattgctaagatatttgcctgatagccgcgctgttttagaaatagatgcgcgtatagcgagtatgaaatcactgttggagagtgatgatggcggtgttcgtcgaactggtccaaacccagcgtatcaagaattaatctctacaatcacgaatgttcaatctgatttgaatgcggcaacgtctcgcgcggcggagttaaaacgtcaggtcaatgaagtgtctcagcgacagaaagaactcgttttgttgcaaccagaatataagaaacttcgccgagagcgtgacgtgttggaaagagcgatggttgaattggcaacgagagagcaatcgaaaaaagcagaagttcagatttcggaaggttcaagcgggagtgttgtaatcgtcgatagggcattaccaccatctaaaggttcgagtatgaagataccgattgttttggcatcagggatgtttgctggatttactgcattaatcgcgggcttacttgttgcatttagtcgtaaaagtctctcaacgcggcgttcaactgaaaagacaatagggttgcctgtcattggtgtgacatcgaagcaataa

SEQ ID NO: 7 - DNA, 1362 bp, unidentified
  source 1..1362: mol_type = genomic DNA
ttgttcaacaacgacacgtgcctttcaaaatggcacaacagcccacattcattaaatggacctctgtactggatcgaacgtgcgttagttttcttcattgttttaacatacgaaacacaaagcatgatagcacctgacccaggaccaatagcgcgggcttctcaggactatggattgcgatactcttaggtagagccgcattggtttcctaaaagctaggaagtgcgagcttaaaattctggcccataattttgttattagcatatctggcattgattgggttattggggctaaatataaggtttagcagtcgcttcggcaaactggtctttagatccaagcctaacacagcggcgtgccattgcacttagcttcagctttatgttcggttgttatcttgcgatacgtgcccccctagtcgacacactaaggatcattggctgggcgtggctaacgatttgtatcctcaattttcttcttatttttggagcccctcacctaggtgtccactcggaattacacgttggagcgtggcgcggtttcatgaccgagaaaaaccatcttggcggtgaaatggcacgcgcaaacctaatattccttgcgcttgtatatttcgatagaaaaacaccagcaggtcagaacaaaaaagcatggtggttaggtctagctttaacgtggatgctcatcttaggttccacatctaaaacggcgctcattgcaacgctaatcccatatttaggttttttattctacagcatcgcgatccgcacaccgatattaggacttttagcgttttggggtggcctgagtttagctggtatcggttacgccataatatccatttcgcctgaaactgttgttgcgctgattgggaaagacctgacgtttaccggtcggacaggcatttgggctatcgttattgatttaatcaatcagcaaaaatggacgggttatggatatggtgcattctgggtcacgcctgatggtccagttgcatttatcgtgaacacactggaatggaacgtgccaacagcccataatggatggctggaagtgggattggctataggataccctggattaatcctgattatctgcatctcactatttgctttaggcaaagctgcatatctggcaacaggcaaacatggcccatttgtctttctcatgctttttcaaataattctattttccttgtcagaaagcatattgatgcaacaaaattcgcatgcatcatccctattctatttcttcactgcttacgcatttattgctcgcaaagtcgcaacagactcacaaacacctctcttaagcgccccgcattgggcgctaccaccgcgtactacgaagaaacgcagctaa

SEQ ID NO: 8 - DNA, 717 bp, unidentified
  source 1..717: mol_type = genomic DNA
atgatgaaaaacaacgcatccatcctcgcactcgctgttctgtcaatatcggcatgctctagccctcaaccgggaccaagcgcgccgacttttcaatcaacgactttctcaaaatggtcgcaaaatgatgcagcctacagattctacccaggcgataagttaaacataactttcagacaagcacctgaattggaccgagaagtcgttattgcaccggatggtcggataagccttccactcatggaccctgtcgttgtcgcggatctttctgctttcgaactacaaaaaattcttgaacgcatctatgctagagaattggtggatccctctctcacagtcacgccagttgaatttgcgtcccaacaaatattcgtcggtggtgaagtcaataatccgggcgtatttccattgccgggccaaattgatccgctgcaagccattgtcttagctggtggctggaacgataattccaaacctgaacaagtcattattttacgtcgcgatcgaaatggtcaaatcatgacccgcgttgttgacgtaaaaaatgctcttcgtgacccaagcaatctagacattggaccattaaaacggtttgacgtcgtgtttgtatcccgcagccgcatagcaaatgaaaacaaatttatacagcaatacgtactatcagcattgccaattgatttttcattcttctacaacctaaaagacaacgccttctaa

SEQ ID NO: 9 - DNA, 711 bp, unidentified
  source 1..711: mol_type = genomic DNA
atgaaagacttacgaaaagatttaactgaaacttggcgtgtcgcaacacgagcaccagtggataatggtggtcgtacgattatgttcatgtctgctatggcaggcgaaggcacatcgagtgtggctgcttcttttgctatgttagcggctcaacgtgcgcgcaagggtgtatggctgattgatctaaatcttatggatggtcggttatttaatgcttttgatcgcggcggcgattttgttgattcatttggtcaggttggtccagctcataacgctgaaatgtctggcgcttcctttttttctatctcaccaccaccaccgccgccaccacctggtaagaagaacgatgcggggctttttgtaatgcatcgtgtggcggatactaaattacttgtgtctaggtttcgaaaagaacgtttagaatcaagcgctcgcgtgcgtgtgaaaacgggagcggactactggaaagccgttagagagatagctgactgggtcattatcgatgcacctgcgttagaaacatcttcagcgggtttggctatctgttcgcaaatggatgcaactgctttagttgtgcgagctgataaaacacctgcgactcaggtgtccaagcttggacaagaaattgaaggccatggtggcacatgtatgggaattgttctcaatgcaaccaaagcagatgcccgattagcggatgaaatttcaggctaa

SEQ ID NO: 10 - DNA, 1250 bp, unidentified
  source 1..1250: mol_type = genomic DNA
atgatgcaagacaatcaagccacccaaatgcctgtccggcgcgaacaacgcggtgtgaatttacgcgttcgtccacgttttggcgtgggcgatatattcttgcaattgtggcgatcactttggattatgatattggtgtttctgcctatcgtgatactgggtgttctgtttgctatgaccatgcccaaaacatatacggcattcggcacaatgcaggtcacgttggataagcaatatatttatgacccgcttgtcggagatgctggacgtggtgtttctattgaaactgaggcaattgttagtgcggaagctgagaaagccaattcccctatattagcgcgtcgcgttatggatcagatggggattggacacatctatcctaaaattgcccaagagatagcagaaacgaatgaccctgacaaaattcgaaaattggaagcctctgcatatgatgctttgaaccaaaattttggtgcatctcacggggtgaaatcacccttgcttacttttgtctttaagcatgaagatccaattgttgccgccgaggttgtcaataaatttctggaacaatttgtcgactatagagaaattcaaagtgatcaagatgatattgcggctgtggctggtcaaagatatcttgtgggtgaggccttaagtgaagccgaatcattgctacgcgacttccttgttgaaaatgaaattggtgaatttgatactgatcgattgtctgttggcacgacattggtgaatttacgcaatgaacttttgaccgttgaggcattggtaaaagaaggtgaaggtcgtttgagtggcttgcgtgctatgctgccaactacgccagaaacgattgcgctagaagttgagacaaatgcatcacaacgtgtgctggacctccagttagagcgtgagcaattattgctaagatatttgcctgatagccgcgctgttttagaaatagatgcgcgtatagcgagtatgaaatcactgttggagagtgatgatggcggtgttcgtcgaactggtccaaacccagcgtatcaagaattaatctctacaatcacgaatgttcaatctgatttgaatgcggcaacgtctcgcgcggcggagttaaaacgtcaggtcaatgaagtgtctcagcgacagaaagaactcgttttgttgcaaccagaatataagaaacttcgccgagagcgtgacgtgttggaaagagcgatggttgaattggcaacgagagagcaatcgaaaaaagc

SEQ ID NO: 11 - DNA, 100 bp, unidentified
  source 1..100: mol_type = genomic DNA
ctataagcaatgaagcattactcatactagccttcttttgaagtgttcatgctaggttgtattgcacaaggtaaatggagacactctgtgttcttcgtga

SEQ ID NO: 12 - DNA, 100 bp, unidentified
  source 1..100: mol_type = genomic DNA
atatatggaaaaagcatcattctggtttgatctatacgtcatcctgatgactgctccctgccttttgggcgactcaaaaacacagcgctaggcatccaat

SEQ ID NO: 13 - DNA, 100 bp, unidentified
  source 1..100: mol_type = genomic DNA
ccacaactgggatcgtcgaagaattgtgccaactgtctaaagcagcaggcgcaacgctttcaacgccaactgaggcagcaagaagctacggcctaatctc

SEQ ID NO: 14 - DNA, 100 bp, unidentified
  source 1..100: mol_type = genomic DNA
gatggtcgaacttatgcttccgcttggtttggttgaagtctcaagaacgggtgtattatcgcttcagcgcggccctcatgcgggagctggtcggtagatt

SEQ ID NO: 15 - DNA, 100 bp, unidentified
  source 1..100: mol_type = genomic DNA
tccataactggtagcattgcgacagcgcacttttttccaataaaggttagaggtctcgccctaaaattatggcgtgcaaccatatatgcggaggctcttc

SEQ ID NO: 16 - DNA, 100 bp, unidentified
  source 1..100: mol_type = genomic DNA
ttcaagtcggcagctaagaaagattgtatttacaagtctctttctatagccagtctaacgagacacttagaatagacagttttaggaacaaatataacag

SEQ ID NO: 17 - DNA, 100 bp, unidentified
  source 1..100: mol_type = genomic DNA
ttaagggctgagattgcacccttgtaagaaggtgcaaaacaattgaatgatcatgctgtattaacctcaacgcatctttccatataacggagcagaaaag

SEQ ID NO: 18 - DNA, 100 bp, unidentified
  source 1..100: mol_type = genomic DNA
catcatattctgattctgctttatctaaacaacttgttcatttgtcgcttgatttgccttaatatttcattatccaaaacggatgcattaagaaagcacc

SEQ ID NO: 19 - DNA, 100 bp, unidentified
  source 1..100: mol_type = genomic DNA
ttgttatttaattaaaaaatatacttttcaaaaagttaacttagtttttctatgttgaaaccaattatagcggcaaaataagatgtagtgaatttgagtg

SEQ ID NO: 20 - DNA, 100 bp, unidentified
  source 1..100: mol_type = genomic DNA
catcatggtgctttcttaatgcatccgttttggataatgaaatattaaggcaaatcaagcgacaaatgaacaagttgtttagataaagcagaatcagaat

SEQ ID NO: 21 - DNA, 100 bp, synthetic construct
  source 1..100: mol_type = other DNA
gatcccgcgaaattaatacgactcactataggggaattgtgagcggataacaattcccctctagaaataattttgtttaactttaagaaggagatatacc

SEQ ID NO: 22 - DNA, 100 bp, synthetic construct
  source 1..100: mol_type = other DNA
tctggtttgatctatacgtcatcctgatgactgctccctgccttttgggcgactcaaaaacacagcgctagtttgtttaactttaagaaggagatatacc

SEQ ID NO: 23 - DNA, 100 bp, synthetic construct
  source 1..100: mol_type = other DNA
caataaaggttagaggtctcgccctaaaattatggcgtgcaaccatatatgcggaggctcttcatgattgatttgtttaactttaagaaggagatatacc

SEQ ID NO: 24 - DNA, 100 bp, synthetic construct
  source 1..100: mol_type = other DNA
ttattctgcaattttatggggatattcccgataagaaaaagcgcaaaaaagttatcaaaagcgcaacttaatttgtttaactttaagaaggagatatacc

SEQ ID NO: 25 - DNA, 100 bp, synthetic construct
  source 1..100: mol_type = other DNA
ttggaaatgtcatttttctcacgcgtggtttgatattatgtgtgacccaagctaatcccaaaataacttaatttgtttaactttaagaaggagatatacc

SEQ ID NO: 26 - DNA, 100 bp, synthetic construct
  source 1..100: mol_type = other DNA
actcacaaacacctctcttaagcgccccgcattgggcgctaccaccgcgtactacgaagaaacgcagctaatttgtttaactttaagaaggagatatacc

SEQ ID NO: 27 - DNA, 100 bp, synthetic construct
  source 1..100: mol_type = other DNA
agcaatacgtactatcagcattgccaattgatttttcattcttctacaacctaaaagacaacgccttctaatttgtttaactttaagaaggagatatacc

SEQ ID NO: 28 - DNA, 100 bp, synthetic construct
  source 1..100: mol_type = other DNA
gcacatgtatgggaattgttctcaatgcaaccaaagcagatgcccgattagcggatgaaatttcaggctaatttgtttaactttaagaaggagatatacc

SEQ ID NO: 29 - DNA, 100 bp, synthetic construct
  source 1..100: mol_type = other DNA
gtctctcaacgcggcgttcaactgaaaagacaatagggttgcctgtcattggtgtgacatcgaagcaataatttgtttaactttaagaaggagatatacc

SEQ ID NO: 30 - DNA, 100 bp, synthetic construct
  source 1..100: mol_type = other DNA
ttcctcctttcagtaatacgactcactataggggaattgtgagcggataacaattcccctctagaaataattttgtttaactttaagaaggagatatacc

SEQ ID NO: 31 - protein, 255 aa, unidentified
MIDWHYTPSRTLPAKLKRRMTQWRHAAPVDVSNTQFHVSYTFDDFPMSAVNGADILESHDGHAAFYACTKMIGTHGAYGDMYDIKTMLDLENRGHEIGAHTHSHLDCAQSKRETVLNDIDANISALMEAGLKKRPTSFAYPYGETLFDTKKEVFKKFDLCRGILPGINVGKVDLAQLRCFELNENPATRIRAINAIEEAGKTGGWVIIFTHDVSPQPTAYGTTTGIVEELCQLSKAAGATLSTPTEAARSYGLIS

SEQ ID NO: 32 - protein, 271 aa, synthetic construct
MHHHHHHMIDWHYTPSRTLPAKLKRRMTQWRHDAPVDVSNTQFHVSYTFDDFPMSAVNGADILESHDGHLAFYACTKMIGTHGAYGDMYDIKTMLDLENRGHEIGAHTHSHLDCAQSKRETVLNDIDANISALMEAGLKKRPTSFKYPYGETLFDTKKEVFKKFDLCRGILPGINVGKLDLAQLRCFELNENPATRIRAINAIEEAGKTGGWVIIFTHDVSPQPTAYGTTTGIVEELCQLSKAAGATLSTPTEAARSYGLISYPYDVPDYA

SEQ ID NO: 33 - DNA, 100 bp, synthetic construct; identical to SEQ ID NO: 21
  source 1..100: mol_type = other DNA

SEQ ID NO: 34 - DNA, 372 bp, synthetic construct
  source 1..372: mol_type = other DNA
gatccggctgctaacaaagcccgaaaggaagctgagttggctgctgccaccgctgagcaataactagcataaccccttggggcctctaaacgggtcttgaggggttttttgatcgacggatcgggagatctcccgatcccctatggttctcagtacaatctgctctgatgccgcatagttaagccagtatctgctccctgcttgtgtgttggaggtcgctgagtagtgcgcgagcaaaatttaagctacaacaagggtacccaaaaaacccctcaagacccgtttagaggccccaaggggttatgctagttattgctcagcggtggcagcagccaactcagcttcctttcgggctttgttagcagccggatc

SEQ ID NO: 35 - DNA, 205 bp, synthetic construct
  source 1..205: mol_type = other DNA
atcgacggatcgggagatctcccgatcccctatggttctcagtacaatctgctctgatgccgcatagttaagccagtatctgctccctgcttgtgtcatgttagacgagactacggcgtatcaattcggtcatagacgagggacgaacagtgttggaggtcgctgagtagtgcgcgagcaaaatttaagctacaacaagggtacc

SEQ ID NO: 36 - DNA, 23 bp, unidentified
  source 1..23: mol_type = other DNA
tttgtttaactttaagaaggaga

SEQ ID NO: 37 - DNA, 48 bp, unidentified
  source 1..48: mol_type = other DNA
ctagcataaccccttggggcctctaaacgggtcttgaggggttttttg

SEQ ID NO: 38 - DNA, 20 bp, unidentified
  source 1..20: mol_type = other DNA
taatacgactcactataggg

SEQ ID NO: 39 - DNA, 816 bp, unidentified
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcat

SEQ ID NO: 40 - DNA, 25 bp, unidentified
  source 1..25: mol_type = other DNA
ggaattgtgagcggataacaattcc

SEQ ID NO: 41 - DNA, 1083 bp, unidentified
  source 1..1083: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 42 - DNA, 717 bp, synthetic construct
  source 1..717: mol_type = other DNA
atgatgaaaaataatgcttctatcctggctctggcggttctgtccatcagcgcatgctcctctccgcaaccgggtccgtctgcaccgacctttcagtctactactttcagcaaatggtctcagaacgatgcggcataccgcttttacccgggcgataagctgaacattaccttccgtcaggcaccggagctggaccgtgaagtggtcatcgcacctgacggtcgtatttctctgccactgatggatccggtagtggtggcagatctgtccgccttcgaactgcagaaaatcctggaacgtatctacgcgcgcgaactggtagatccgagcctgactgttaccccagtggaattcgcctcccagcagatctttgtcggtggcgaggtaaacaacccgggcgttttccctctgccaggtcagatcgatccgctgcaggctatcgtgctggcaggtggctggaacgacaacagcaaaccggaacaggtcattatcctgcgtcgcgatcgcaacggtcagattatgactcgcgttgtggacgtgaaaaacgcgctgcgtgacccgtctaacctggatatcggtccgctgaaacgtttcgatgttgtgttcgtatctcgcagccgcatcgctaacgaaaataaattcatccagcaatatgtgctgtccgcgctgcctatcgacttctctttcttctacaacctgaaggacaacgcgttctaa

SEQ ID NO: 43 - DNA, 1485 bp, synthetic construct
  source 1..1485: mol_type = other DNA
atgatgcaggacaatcaagcaacccagatgccggtacgccgcgaacagcgcggcgttaacctgcgtgtacgtccgcgctttggcgtgggtgatatctttctgcaactgtggcgctccctgtggattatgatcctggttttcctgccgatcgtgatcctgggcgtgctgttcgctatgactatgccgaaaacctacaccgcgttcggcaccatgcaggttaccctggataaacagtacatttatgatccgctggtaggtgacgcgggtcgtggcgtctctattgaaaccgaagcaattgtttccgctgaggcggagaaagcgaactctccgattctggcacgtcgcgttatggatcagatgggtatcggtcacatctaccctaagatcgctcaggaaattgcagaaaccaacgacccggacaaaatccgtaaactggaagcttccgcttacgacgcgctgaaccagaatttcggcgcgtcccacggcgtgaaatctccgctgctgaccttcgtcttcaaacacgaagacccgatcgttgcggctgaggttgtgaacaaattcctggaacaattcgttgattaccgcgagatccaatccgaccaagatgacatcgcggctgttgccggtcagcgttacctggttggcgaagctctgagcgaagctgaatctctgctgcgtgatttcctggttgagaacgaaattggtgagtttgacaccgaccgtctgtctgttggtaccactctggtgaacctgcgcaacgaactgctgactgttgaagccctggtaaaggaaggtgaaggtcgtctgtccggtctgcgcgccatgctgccgacgactccggaaactattgcactggaagtcgagaccaatgcgtctcagcgtgtgctggatctgcagctggaacgtgaacagctgctgctgcgttatctgccagattcccgcgcggtactggaaattgacgcacgtatcgcatccatgaaaagcctgctggagagcgacgatggcggcgttcgtcgtactggcccaaacccagcataccaggaactgatcagcactatcacgaacgtacagtccgatctgaatgctgcgacgtcccgtgcggcagaactgaaacgtcaggttaacgaggtgtcccagcgccagaaggaactggtactgctgcagccagaatacaaaaaactgcgccgtgaacgtgatgtgctggaacgcgctatggttgaactggctacccgtgagcagagcaaaaaagcggaagttcagatctccgaaggttccagcggttccgtagttatcgtggatcgtgctctgccgccgtctaaaggtagctctatgaaaattccgatcgtcctggcgtctggcatgtttgctggtttcaccgcgctgattgctggtctgctggttgctttcagccgtaaatccctgtctacccgccgctctactgaaaagactatcggtctgccggtgatcggcgttacctccaaacag

SEQ ID NO: 44 - DNA, 708 bp, synthetic construct
  source 1..708: mol_type = other DNA
atgaaagacctgcgtaaagacctgactgaaacctggcgtgtagcaacgcgtgcacctgttgataacggtggccgtacgatcatgttcatgagcgccatggctggcgaaggtacctcttctgtggctgcgagctttgctatgctggcggctcagcgtgctcgtaaaggtgtctggctgatcgatctgaacctgatggacggccgcctgtttaatgccttcgaccgtggcggtgactttgtggacagctttggtcaggtgggtccagcgcacaacgcagaaatgtccggcgcgtccttcttcagcatctctcctccgccgccgccgccgcctccgggtaaaaaaaacgacgccggcctgttcgtaatgcaccgtgttgcggacacgaaactgctggtgtctcgtttccgcaaagagcgtctggaaagcagcgcacgtgtccgtgtaaaaaccggcgcggactattggaaagcagttcgtgagatcgctgactgggttatcatcgatgcgccggctctggagacctcttctgcgggtctggcaatttgttctcagatggacgcgaccgcgctggttgtgcgtgcggacaaaacgccagctacccaggtatccaagctgggccaagagatcgaaggtcacggtggtacctgcatgggcatcgtactgaacgcaaccaaagcggatgcacgtctggcagacgagatttctggc

SEQ ID NO: 45 - DNA, 990 bp, synthetic construct
  source 1..990: mol_type = other DNA
atgaacactaccccacagctgagcgccaacccaccagtacgcgatacgactactccacgtgagatcgttgttgacaacccgcattggaaagacaaggccaaccagaaactgtccattctggttccgtcctacaaagacgacccggaagcactgctggctagcctgtctaaatgcaaaaacgtggaagacatcgaattcatcctgtacgatgatggcggtggcgaccgtacgctgatcgaaaaaattagcgcgcacgctgcagaggcttctttcccagttcgcatcatctctgcgcaacataacattggtcgtgcgggcgctcgtaaccgtctgctgaactacgctcgctgcaaatgggtactgctgctggacgctgacatgctgccggacaatgaagacttcctgaccaactacatccaggaaatctccgttcaccctgatcctaaactgatcgtgggcggtttttccctgaaacaggcatccactgctccggaatacgccctgcaccgttggcaggcagaaaaaagcgaatgcatgtcttccaaactgcgcaacaccgaaccgggccgctacgtcttcacttccaacgtgctggcgcacgcagatctgttcgaagaggtaccgttcgacgaaacgtttagcggttggggttgggaagacgtagactggggcctgcgcatcgcaaagacttacctggttctgcatatcgacaacactgctacccacctgggcctggataccgaccgtgatctgatgaaaaagtacggcaagtctggcgcgaacttcaaaaaagcaatcgaaaagcacccagatgcactgaaaagcacgagcctgtataaggttgcaaacaagctggcaccgatcccgtttaaacctgtcatcaaatctatcacgggcagcatcgcgactgcccacttcttcccgatcaaagtccgtggcctggcgctgaaactgtggcgcgcgacgatctacgctgaagcactgcacgac

SEQ ID NO: 46 - DNA, 990 bp, synthetic construct
  source 1..990: mol_type = other DNA
atgaacaccaccccacaactgtccgcgaacccgcctgtacgtgataccactactccgcgtgaaatcgtagtcgacaatccgcactggaaagacaaggccaaccaaaaactgtccattctggttccatcctacaaagatgatccggaagcactgctggcttctctgtccaaatgtaagaacgtggaggacatcgagttcattctgtacgacgatggcggcggcgaccgtactctgatcgaaaaaatctctgcgcacgccgcagaggcctctttcccggtgcgcattatttccgcgcagcataacattggtcgtgctggtgcgcgcaaccgtctgctgaactatgcgcgctgtaagtgggtactgctgctggatgcggacatgctgcctgacaacgaagactttctgaccaattacatccaggagatctctgtgcacccggatccaaaactgatcgtgggcggtttctctctgaaacaggcttccactgcaccagaatacgcactgcatcgttggcaggccgagaaatctgaatgcatgtcttctaagctgcgcaacactgagccgggccgttacgtttttactagcaacgtcctggcacatgctgacctgtttgaagaggtgccgttcgacgaaactttctctggttggggttgggaagacgttgattggggtctgcgtatcgcgaagacctacctggtactgcacattgacaacactgctacgcacctgggtctggacactgatcgtgatctgatgaaaaaatatggcaagtctggcgcgaactttaaaaaagccatcgagaaacacccggacgccctgaagtccactagcctgtacaaggtggcaaacaaactggctccgattccgttcaaaccggtgattaaatccatcaccggtagcatcgcgaccgctcacttcttcccgattaaggtgcgcggcctggcactgaaactgtggcgtgcgacgatctatgccgaagctctgcacgat

SEQ ID NO: 47 - DNA, 990 bp, synthetic construct
  source 1..990: mol_type = other DNA
atgaatactaccccacagctgtctgcaaacccacctgtgcgcgatactaccaccccgcgtgagatcgtggtggataatccacactggaaagataaggcgaaccagaaactgagcatcctggtgccgtcttataaagatgacccagaagcactgctggccagcctgtctaagtgcaaaaatgtcgaagacatcgaattcattctgtatgatgacggcggtggtgaccgtaccctgattgaaaaaatctctgcgcacgcggcggaagcctccttcccggttcgcattatctccgcgcaacacaatatcggccgcgctggtgcccgtaaccgtctgctgaactatgcacgttgtaaatgggtcctgctgctggatgcggatatgctgccggacaacgaagatttcctgacgaactacattcaggaaatttccgtgcatccagatccaaagctgattgtaggtggcttcagcctgaaacaggcttctaccgcgccggaatatgcactgcatcgttggcaggcggaaaaaagcgaatgcatgtcttccaagctgcgtaacacggaaccgggtcgttacgttttcacttccaacgttctggcacacgccgatctgttcgaggaggtgccattcgatgaaaccttcagcggctggggttgggaagatgttgactggggtctgcgtattgcgaaaacctacctggttctgcacattgacaacaccgcaactcacctgggcctggacaccgatcgcgacctgatgaaaaagtacggcaaatctggtgcgaacttcaaaaaagcgatcgaaaagcatccggacgcgctgaaatctacgtctctgtacaaagtcgctaacaaactggcgccgattccgtttaaaccggtcatcaaatccatcaccggttctattgcaacggctcacttcttcccgattaaagttcgtggcctggctctgaaactgtggcgtgccactatctatgcggaagcactgcacgac

SEQ ID NO: 48 - DNA, 1359 bp, synthetic construct
  source 1..1359: mol_type = other DNA
ctgttcaacaacgatacctgtctgtctaaatggcacaactctccgcactccctgaacggcccactgtactggattgagcgtgctctggttttcttcatcgtgctgacttacagcggcctgtggattgcaatcctgctgggtcgtgcagcggaaacgcagtctatgatcgcgccagacccgggtccgattgctcgtgccagctggttcccggcgtatctggcactgatcggtctgctgggcctgaatattaaaaaactgggttctgcgtccctgaaattctggccaatcatcctgctgctgggtctggcggttgcgagcgcgaactggtctctggatccttctctgacccagcgtcgtgctattgccctgagcttctccttcatgttcggttgctatctggctatccgtgctccgctggtagacaccctgcgtattatcggctgggcctggctgacgatctgcattctgaactttctgctgatcttcggcgctcctcacctgggtgttcattccgaactgcacgttggcgcgtggcgcggcttcatgactgagaaaaaccatctgggcggtgaaatggcccgtgctaacctgatcttcctggctctggtctatttcgaccgtaagaccccggcaggccagaacaaaaaagcatggtggctgggcctggcgctgacctggatgctgatcctgggttccacctccaaaacggccctgatcgcgacgctgatcccgtatctgggttttctgttctacagcatcgcaatccgtactccgatcctgggtctgctggcgttctggggtggcctgtctctggctggcatcggttacgcaatcatcagcatcagcccagaaaccgtggtggcactgatcggcaaggacctgactttcactggtcgcaccggtatctgggcgatcgttatcgacctgatcaatcagcagaaatggacgggctatggctacggtgctttctgggtgaccccggatggcccggtagctttcattgtcaacaccctggaatggaacgtgccaaccgctcacaacggttggctggaagtgggcctggcgattggctatccgggcctgattctgatcatctgcatctctctgttcgctctgggtaaagccgcctacctggctaccggcaaacacggtccgttcgttttcctgatgctgttccagatcattctgttctccctgtccgaaagcatcctgatgcagcagaacagccacgcatcttctctgttctacttcttcaccgcttacgcttttatcgcacgtaaagtggcgaccgactcccagactccgctgctgtctgcgccgcactgggcactgcctccgcgtactaccaaaaaacgtagc

SEQ ID NO: 49 - DNA, 708 bp, synthetic construct
  source 1..708: mol_type = other DNA
atgaaagacctgcgtaaagacctgactgaaacttggcgtgtggcgactcgtgcaccggttgacaatggtggtcgtaccatcatgttcatgtccgccatggctggcgaaggtacctccagcgtggcagcgtcctttgcgatgctggctgctcagcgtgcgcgcaaaggtgtttggctgatcgatctgaacctgatggacggtcgtctgttcaacgcgtttgaccgcggtggtgatttcgtagactctttcggtcaggtcggcccggctcataacgctgagatgagcggtgcttctttcttttccatttctccgccaccaccgccgccaccgccaggcaaaaagaacgatgcaggcctgttcgtgatgcaccgtgttgcggacaccaaactgctggtgagccgcttccgtaaagaacgtctggaaagcagcgcgcgcgtccgtgtgaaaaccggtgcggactactggaaggctgttcgtgaaatcgctgactgggtgatcattgatgccccggctctggaaacctcctctgcaggtctggctatctgctcccagatggatgctaccgctctggttgtgcgtgcagataaaaccccagcgacccaggtaagcaaactgggccaggagatcgaaggtcacggtggcacttgcatgggcattgtgctgaacgcgaccaaagcggatgcacgtctggcagatgaaatcagcggc

SEQ ID NO: 50 - DNA, 1443 bp, synthetic construct
  source 1..1443: mol_type = other DNA
atgatttggcgccacctgttcggctacctgcctgtaaacgttatccagggcctggtgtccttcggtgcggtatacgctttcactcgcctgctgggcgatgacggctacggcagctatgctctggttctgaccatcatgtccgcaagccacaccaccacgctgacttggactgaagcagcagcgtatcgtttcgcgggtgaagcgcagagcaaaggtggcatgaatgaccacatccgtacgagcatctacctggcgctgttcagcctgatcccagctctgctgattgtggcatgcggctggatggcatccgaaaacaacccgaacatgcaggcggcaatcatctggctggctctgtctatgccgtgcctgtctattatccaaatgagcctggaaattcacaaggcgcgtcagcaggtttcccgtttcgcaaaggtctctatcgcccacgctctgactggtttctgtggtggcctgtatttcgcatctcagactgatgcaggtgcggcagcgccttttatgggcctggctctgtccggtgtaatctttgcaagcgtgcagggcctgttcctgtggaaagaatctaaagacggttctttccagatggtacgtgcgaaacgttactttgcttacggcatgccgctggctctggctctgctgctggaaattgcactgtctgcgtccgatcgttttctgatcgcctacttcatcgataacgcagctgttggtgcgtatgctgccggttatggcgtgagcgatcagtccatccgtctgctgtgcatgtggggtgcgatggctggtgcgcctctgctgatggaatcttacgagaaacacggtctgaacggcatcgaagaaccgggtaaagcaatgatccgtatgctgatgctgatcgcgtttccggccgcaaccggtctggcgatggtggccgaaccgctggcacagtttatgatcggtgaggagctgcgtgatcaggcgaaacacaccattccgtggattgcactggccggcctgatgaacggtctggtgatctattacttctccgaatccttccagctggcacgtaaaaccgccctgcgtgcatctctgatgctgatcccggccatcctgaacgtaatcctgaacatcatcctgctgcctaaaatgggcctgatgggtgctgtatatgcgactgttatttgttacggcgttgcgctgattatcatcatgggtgtaggtcgtcgtttcatcccgctgccggttccgatgaaagacattgtgctgatcgccattgcgtgtgcgggtatggcgagcatcgtttacatcctgccgcaaatcggtggcttcccggaactgatgctgaaggccatcgtaggcggtatcatctatggtgtactggcaatcgtactgaacgcagctggtgccaaagatctgattaaagcgctgaaagaccgtaaaaacgctacccag

SEQ ID NO: 51 - DNA, 744 bp, synthetic construct
  source 1..744: mol_type = other DNA
atgagctctggtaaaaagatctacgacatgctggaacacctgactgtacctgaaaccgaagccaacgttgacgcactgctgaatggtctgccggacgctacttctccgcaggttatcagcttcgttaacgctcacgcagtaaacctgatggtgaaagacgaaggtctgttcaaagcgctgatcggttctgacatcctgctgcgcgacggctctggcatgaaaatcctgatgaagtggctgaaccaaaacccgggtgcaaacctgaacggcaccgatctgattccgcgtattatcgagaagttcgatggtatgaaagtggcggttttcggtacccaagaaccatggctgtctaaaggttgcgacgtaattgaaacccgtggcggcaccatcgtgtcccgtctgaacggtttccaagatgaagccgcttacattgaggcgatcgaaacctccaaaccggacctggtaatcctggctatgggtatgccgaaacaggaaatgacgtctatggcactgcgtgcggccgcctcttggccaaccaccattgtcaacggtggtgcaatcatcgatttcctggcggaacgcgtaaaccgtgctccggaaacttggcgtaaactgggtatggaatggctgtaccgtctgattcaagagccgaaacgcctgttcggtcgctacgtagtcggcaacgttatcttcctgacgcgtggtctgattctgtgcgttactcaagcgaacccgaaaatcacc